(19) United States
(12) Patent Application Publication
(10) Pub. No.: US 2013/0289399 A1
(43) Pub. Date: Oct. 31, 2013

(54) DECOMPRESSION SYSTEMS AND METHODS OF USING THE SAME

(71) Applicant: VERTIFLEX, INC., San Clemente, CA (US)

(72) Inventors: Andy W. Choi, Irvine, CA (US); Arthur C. Kwun, Newark, CA (US); Martin A. Leugers, San Francisco, CA (US); Graham R. Faulknor, San Mateo, CA (US); Shigeru Tanaka, Half Moon Bay, CA (US); David J. Rinaldis, Redwood City, CA (US); Adam Beckett, San Clemente, CA (US)

(73) Assignee: Vertiflex, Inc., San Clemente, CA (US)

(21) Appl. No.: 13/844,173

(22) Filed: Mar. 15, 2013

Related U.S. Application Data
(60) Provisional application No. 61/755,329, filed on Jan. 22, 2013.
(60) Provisional application No. 61/745,470, filed on Dec. 21, 2012.
(60) Provisional application No. 61/639,828, filed on Apr. 27, 2012.

(51) Int. Cl. A61B 17/16; A61B 17/88; A61M 5/00

(52) U.S. Cl. CPC A61B 17/1671; A61M 5/007; A61B 17/88
USPC 600/431; 606/86 R; 606/80; 606/79

(57) ABSTRACT

A method for treating spinal nerve compression includes positioning a media delivery device between a first vertebra and a second vertebra. A visualization media can be delivered from an outlet of the media delivery device while the outlet to the media delivery device is positioned outside of an epidural sac of the subject. The media delivery device can be spaced apart from a portion of the epidural sac between a spinal cord and a ligamentum flavum. A series of instruments can be used to perform a decompression procedure on the subject.

### CROSS-REFERENCE TO RELATED APPLICATIONS

This application claims priority to U.S. Provisional Application No. 61/755,329, filed Jan. 22, 2013, U.S. Provisional Application No. 61/745,470, filed Dec. 21, 2012, and U.S. Provisional Application No. 61/639,828, filed Apr. 27, 2012. Each of these applications is incorporated by reference in its entirety.

### TECHNICAL FIELD

The present disclosure relates generally to medical systems and, more particularly, to systems, devices, and methods for treating spinal nerve compression.

### BACKGROUND

Spinal nerve compression can be caused by narrowing of the spinal canal associated with arthritis (e.g., osteoarthritis) of the spine, degeneration of spinal discs, and thickening of ligaments. Arthritis of the spine often leads to the formation of bone spurs which can narrow the spinal canal and press on the spinal cord. In spinal disk degeneration, inner tissue of the disk can protrude through a weakened fibrous outer covering of the disk. The bulging inner tissue can press on the spinal cord and/or spinal nerve roots. Ligaments located along the spine can thicken over time and press on the spinal cord and/or or nerve roots. Unfortunately, spinal nerve compression can cause lower back pain, hip pain, and leg pain and may also result in numbness, depending on the location of the compressed nerve tissue. In the lower back, spinal stenosis may lead to spinal cord compression and numbness of the legs.

### BRIEF DESCRIPTION OF THE DRAWINGS

FIG. 1 is a side view of a spinal nerve decompression system in accordance with an embodiment of the disclosure.

FIG. 2 is a side view of a bone removal device between two vertebrae in accordance with an embodiment of the disclosure.

FIG. 3 is an isometric view of an instrument positioner apparatus configured in accordance with an embodiment of the disclosure.

FIG. 4 is a top plan view of the instrument positioner apparatus of FIG. 3 in accordance with an embodiment of the disclosure.

FIG. 5 is a cross-sectional view of the instrument positioner apparatus of FIG. 4 taken along a line 5-5.

FIGS. 5A-5C illustrate the instrument positioner apparatus holding an instrument in different positions.

FIG. 6 is an isometric view of a tissue removal instrument configured in accordance with an embodiment of the disclosure.

FIG. 7 is an enlarged view of a bone removal device of the tissue removal instrument of FIG. 6 in accordance with an embodiment of the disclosure.

FIG. 8 is an isometric view of an instrument configured in accordance with an embodiment of the disclosure.

FIG. 9 is a side view of the instrument of FIG. 8 in accordance with an embodiment of the disclosure.

FIG. 10 is an isometric view of a tissue removal device configured in accordance with an embodiment of the disclosure.

FIG. 11 is a flow chart illustrating a method for reducing spinal nerve compression in accordance with an embodiment of the disclosure.

FIG. 12 is a top view of vertebrae and a media delivery device positioned to deliver visualization media in accordance with an embodiment of the disclosure.

FIG. 13 is an enlarged view of tissue adjacent to a vertebral foramen and a distal end of the media delivery device of FIG. 12.

FIG. 14 is an isometric view of a cannula positioned between two vertebrae in accordance with an embodiment of the disclosure.

FIG. 15 is an isometric view of a partially assembled instrument positioner apparatus in accordance with an embodiment of the disclosure.

FIG. 16 is an isometric view of an assembled instrument positioner apparatus in accordance with an embodiment of the disclosure.

FIG. 17 is an isometric view of a spinal nerve decompression system having an instrument positioner apparatus and a tissue removal instrument ready to remove tissue in accordance with an embodiment of the disclosure.

FIG. 18 is a cross-sectional view of the spinal nerve decompression system of FIG. 17 in accordance with an embodiment of the disclosure.

FIG. 19 is a side view of an implanted device in accordance with an embodiment of the disclosure.

FIG. 20 is an isometric view of an instrument configured in accordance with another embodiment of the disclosure.

FIG. 21 is an enlarged isometric view of a distal tip of the instrument of FIG. 20 in accordance with an embodiment of the disclosure.

FIG. 22 is a side view of the distal tip of the instrument of FIG. 20 in accordance with an embodiment of the disclosure.

FIG. 23 is a top plan view of the distal tip of the instrument of FIG. 20 in accordance with an embodiment of the disclosure.

FIGS. 24 and 25 are isometric and side views of an instrument configured in accordance with another embodiment of the disclosure.

FIG. 26 is an isometric view of a tissue removal instrument in accordance with an embodiment of the disclosure.

FIG. 27 is an isometric exploded view of the tissue removal instrument of FIG. 26 in accordance with an embodiment of the disclosure.

FIG. 28 is a cross-sectional view of the tissue removal instrument of FIG. 26 in accordance with an embodiment of the disclosure.

FIGS. 29A and 29B are detailed cross-sectional views of a depth stop mechanism in accordance with an embodiment of the disclosure.

FIG. 30 is a detailed cross-sectional view of a jaw assembly in accordance with an embodiment of the disclosure.

FIG. 30A is an isometric view of a jaw assembly in an open position in accordance with an embodiment of the disclosure.

FIG. 30B is a top view of the jaw assembly of FIG. 30A.

FIG. 30C is a side view of the jaw assembly in an open position.

FIG. 30D is a side view of the jaw assembly in a closed position.

FIG. 30E is a cross-sectional view of the jaw assembly in the open position.

FIG. 31 is a side view of a debulker instrument in accordance with an embodiment of the disclosure.

FIG. 32 is a side view of the debulker instrument of FIG. 31 with a portion of a housing shown removed.

FIG. 33 is a side view of the debulker instrument of FIG. 31 with a tool ready to be installed.

FIG. 33A is an isometric view of a distal portion of the debulker instrument of FIG. 31.

FIG. 33B is a top view of the distal portion of the debulker instrument of FIG. 31.

FIG. 33C is a side view of the distal portion of the debulker instrument of FIG. 31.

FIG. 34 is an isometric view of a reamer instrument in accordance with an embodiment of the disclosure.

FIG. 35 is a cross-sectional view of the reamer instrument of FIG. 34.

FIG. 36 is a detailed cross-sectional view of the reamer instrument of FIG. 34.

FIG. 37 is an isometric view of a tissue removal instrument in accordance with an embodiment of the disclosure.

FIG. 38 is an isometric view of a reamer instrument in accordance with an embodiment of the disclosure.

FIG. 39 is a cross-sectional view of the reamer instrument of FIG. 38.

FIG. 40 is an isometric view of a cannula in accordance with an embodiment of the disclosure.

FIG. 41 is a cross-sectional view of the cannula of FIG. 40.

FIGS. 42 and 43 are end views of the cannula of FIG. 40.

FIG. 44 is an isometric view of an instrument positioner apparatus in a closed configuration in accordance with an embodiment of the disclosure.

FIGS. 45 and 46 are isometric views of the instrument positioner apparatus of FIG. 44 in an open configuration.

FIG. 47 is a top view of the instrument positioner apparatus of FIG. 43.

FIG. 48 is a cross-sectional view of the instrument positioner apparatus taken along line 48-48 of FIG. 47.

FIG. 49 is an isometric view of a collar in accordance with an embodiment of the disclosure.

FIG. 50 is a top view of the collar of FIG. 49.

FIG. 51 is a detailed view of a portion of the collar of FIG. 49.

FIG. 52 is a cross-sectional view of the collar taken along line 52-52 of FIG. 50.

FIGS. 53-55 illustrate a method of assembling an instrument positioner assembly in accordance with an embodiment of the disclosure.

FIGS. 56-58 illustrate the instrument positioner assembly holding a cannula in a patient in accordance with an embodiment of the disclosure.

### DETAILED DESCRIPTION

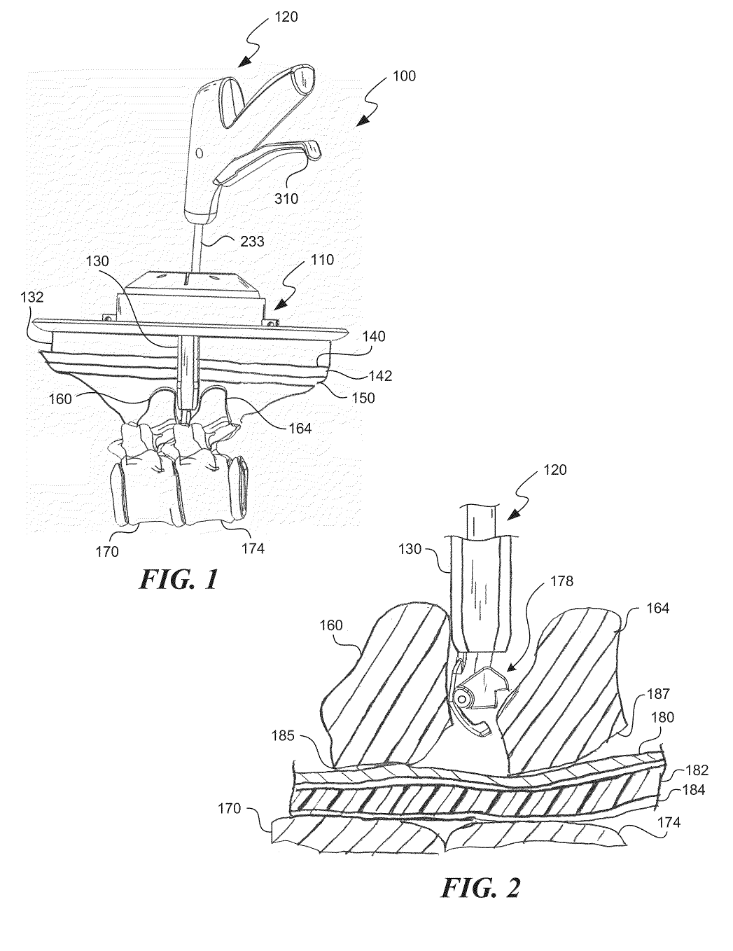

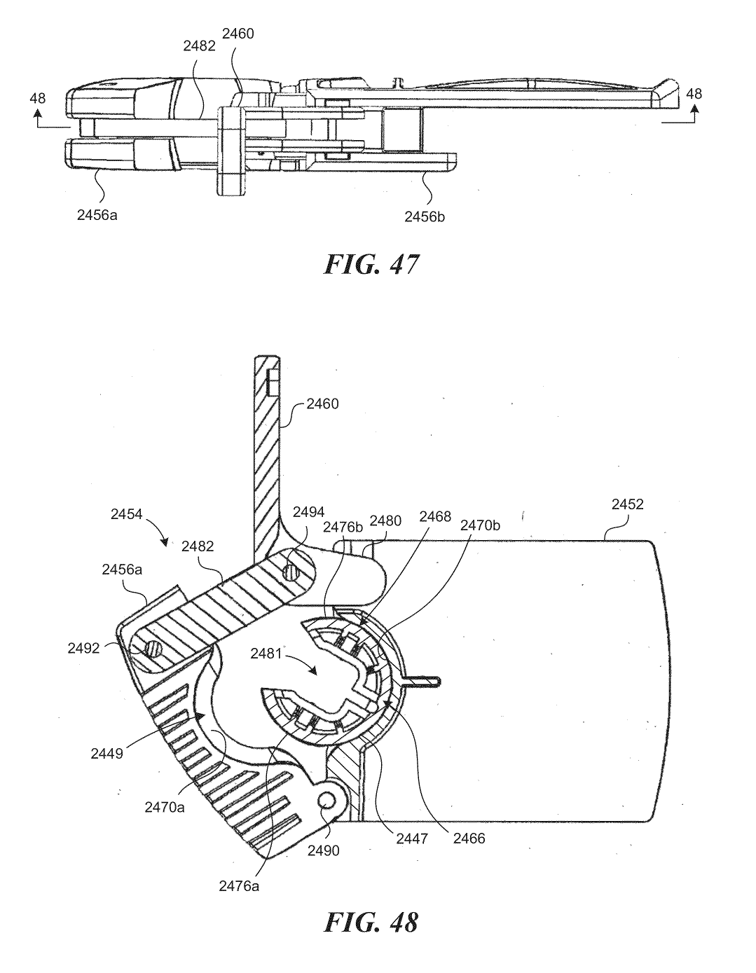

The following disclosure describes various embodiments of medical systems and devices and associated methods of use. At least some embodiments of a treatment system include an instrument positioner apparatus for providing access to a treatment site. A series of instruments can be delivered via the instrument positioner apparatus and used to alter (e.g., crush, separate, cut, debulk, break, fracture, remove, or the like) tissue. Visualization procedures can be used to position the instruments to prevent or limit injury or damage to non-targeted tissues. Certain details are set forth in the following description and in FIGS. 1-58 to provide a thorough understanding of such embodiments of the disclosure. Other details describing well-known structures and systems often associated with, for example, treating the spine, spinal nerves (e.g., nerves in the spinal cord, nerves in nerve roots exiting the spinal cord, etc.), or removing tissue are not set forth in the following description to avoid unnecessarily obscuring the description of various embodiments of the disclosure.

### A. Overview

At least some embodiments are directed to systems for treating spinal nerve compression or other conditions of a human subject. One method includes positioning a media delivery device between a first vertebra and a second vertebra. A visualization media can be delivered from an outlet of the media delivery device while the outlet to the media delivery device is positioned outside of an epidural sac of the subject. The media delivery device can be spaced apart from a portion of the epidural sac between a spinal cord and a ligamentum flavum. In some procedures, tissue is removed from a treatment site using an instrument while viewing both the instrument and the visualization media. The visualization media can also be used to perform other procedures. The system can be used to perform decompression procedures (e.g., posterior lumbar decompressive procedures).

In some embodiments, a method for treating spinal nerve compression comprises delivering a visualization media from an outlet of a media delivery device positioned within a dural sac of a subject such that the visualization media is retained within the dural sac and contacts the spinal cord. A tissue removal instrument is used to remove tissue (e.g., bone tissue, ligament tissue, etc.) from the target site. Any number of instruments can be used to, for example, cut tissue, loosen tissue, crush bone, or otherwise alter the treatment site. In some procedures, tissue can be removed from one or more lateral recesses of a vertebra. In other procedures, tissue can be removed from other features or anatomical structures proximate to the spinal cord, the vertebra, or other locations along the spine or other treatment sites.

In certain embodiments, a method for treating spinal nerve compression comprises delivering a visualization media to a patient. Tissue is removed using a tissue removal instrument while viewing the tissue removal instrument and the visualization media. Visualization techniques (e.g., fluoroscopy) can be used to view at least a portion of the tissue removal instrument (e.g., a distal tip of the tissue removal instrument proximate to the treatment site) and at least some of the visualization media. The tissue removal instrument can be positioned using the visualization media to identify targeted features (e.g., tissue to be removed) and non-targeted features. The method can further include, in some embodiments, delivering a spinal device, such as a spinal implant, a spacer device, prosthetics disk, or the like. In some procedures, fluoroscopy (e.g., anterior-posterior imaging, lateral imaging, contralateral-oblique imaging, etc.) is used to view the treatment site, tools, and delivery path. In certain procedures, visualization techniques can be used to identify margins of the epidural space, dura, ligamentum flavum, and/or nerve roots relative to the lamina and interlaminar space, as well as the features of instruments. Contrast media can be refreshed to maintain desired imaging.

At least some embodiments are directed to a surgical instrument. The surgical instrument can comprise a tool and a handle assembly. The tool can comprise a main body and a distal portion. The handle assembly can include a handle and a depth stop mechanism. The handle assembly can be configured to be manually gripped by a user. The depth stop mechanism can be manually moved to adjust the depth of penetration of the distal portion of the tool. The surgical instrument can be, without limitation, a tissue removal instrument, a debulker instrument, a reamer instrument, or other type of instrument.

The distal portion of the tool can include, without limitation, a jaw assembly, a reamer, one or more cutting edges, one or more blades, combinations thereof, or the like. The depth stop mechanism and the tool can cooperate to limit the depth of penetration of the tool to, for example, prevent or inhibit contacting of non-targeted tissue (e.g., tissue at or proximate to the treatment site). The depth stop mechanism, in some embodiments, includes a locking assembly and a stop member. The locking assembly can have a locked configuration for holding the stop member and an unlocked configuration for moving the stop member.

The stop member, in some embodiments, can be positioned at numerous discrete positions to define corresponding depths of penetration. In a single procedure, the stop member can be moved to different positions to target different sites. In some embodiments, the stop member includes, without limitation, a head that surrounds the main body of the tool. The head can be moved axially along a longitudinal axis of the tool. The head can contact a stop located in a delivery device to, for example, prevent distal movement of the instrument. The stop can be a proximal surface of a cannula, a shoulder of a cannula, a delivery instrument

The locking assembly has a control element movable from a first position to a second position to allow movement of the depth stop mechanism from the locked configuration to the unlocked configuration. In some embodiments, the control element can be moved from an undepressed position to a depressed position to unlock the depth stop mechanism. The unlocked depth stop mechanism can be reconfigured or moved to adjust the depth of penetration of the distal portion of the tool. In some embodiments, a biasing element can urge the locking mechanism towards the locked configuration. When the user overcomes the biasing force provided by the biasing element, the depth stop mechanism can be moved from the locked configuration to the unlocked configuration. When the user releases the control element, the locking assembly can be returned to the locked configuration by the biasing element.

In some embodiments, a system comprises an instrument positioner assembly that includes a base and a joint device configured to rotatably couple a cannula to the base such that an end of the cannula is positionable generally between a first spinous process of a first vertebra and a second spinous process of a second vertebra. In one embodiment, the base is configured to be positioned on a human subject. The base can be a plate, a platform, or other stabilizing structure. The joint device can include, for example, a joint that provides two degrees of freedom, three degrees of freedom, or the like. For example, the joint device can be a ball or collar and socket joint.

### B. Decompression Systems

FIG. 1 is a side view of a spinal nerve decompression system 100 (“system 100”) that includes an instrument positioner apparatus 110 (“positioner apparatus 110”) and a tissue removal instrument 120 (“instrument 120”) in accordance with an embodiment of the disclosure. The positioner apparatus 110 includes a cannula 130 extending through a subject's skin 140, subcutaneous tissue 142, and a supraspinal ligament 150. The positioner apparatus 110 can hold the cannula 130 at different positions and orientations to allow convenient access to a wide range of treatment sites.

FIG. 2 is a detailed side view of a tissue removal device 178 of the instrument 120 of FIG. 1 positioned between the spinous processes 160, 164 of the vertebrae 170, 174, respectively. The vertebrae 170, 174 are shown in cross section in FIG. 2. The tissue removal device 178 is spaced apart from a ligamentum flavum 180. A spinal cord 182 is positioned between the ligamentum flavum 180 and a ligament 184. The spinal cord 182 extends from the brain to the bottom of the spine and extends through vertebral foramina 185, 187. Spinal nerves branch from the spinal cord 182 and exit the spine and extend to other parts of the body. Visualization media can be used to image various features (e.g., anatomical structures, targeted tissue, non-targeted tissue, or the like), including the ligamentum flavum 180, spinal cord 182, nerves branching from the spinal cord 182, ligament 184, vertebrae 170, 174, or any other features or anatomical structures of interest while the tissue removal device 178 removes bone from the vertebra 174. In some embodiments, the tissue removal device 178 is prevented or otherwise inhibited from contacting the spinal cord 182 to inhibit, limit, or substantially prevent damage and/or injury to the spinal cord 182. For example, the motion of the tissue removal device 178 can be restricted to maintain a margin between the tissue removal device 178 and the spinal cord 182.

Referring to FIGS. 1 and 2 together, the instrument 120 can be replaced with any number of different instruments to treat a wide range of symptoms, conditions, and/or diseases, including, without limitation, spinal nerve compression (e.g., spinal cord compression, spinal nerve root compression, or the like), spinal disk herniation, osteoporosis, stenosis, or other diseases or conditions. In one embodiment, the system 100 is used to perform a spinal cord decompression procedure that includes, without limitation, delivering visualization media, removing bone from one or both vertebrae 170, 174, separating the ligamentum flavum 180 from one or both vertebrae 170, 174, cutting or debulking the ligamentum flavum 180, and removing loose tissue. Each stage of the procedure can be performed with a different instrument.

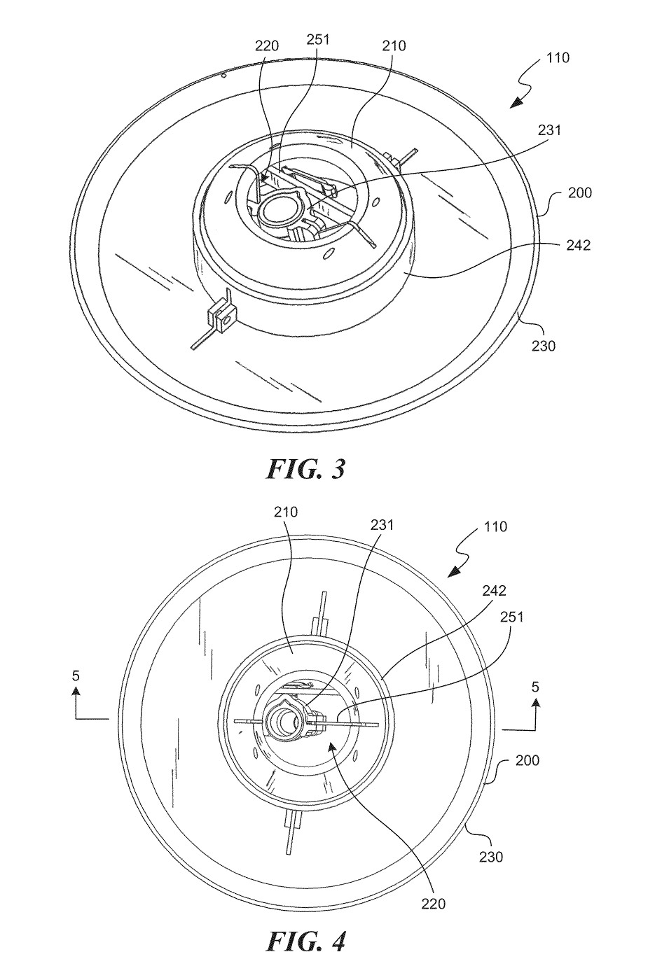

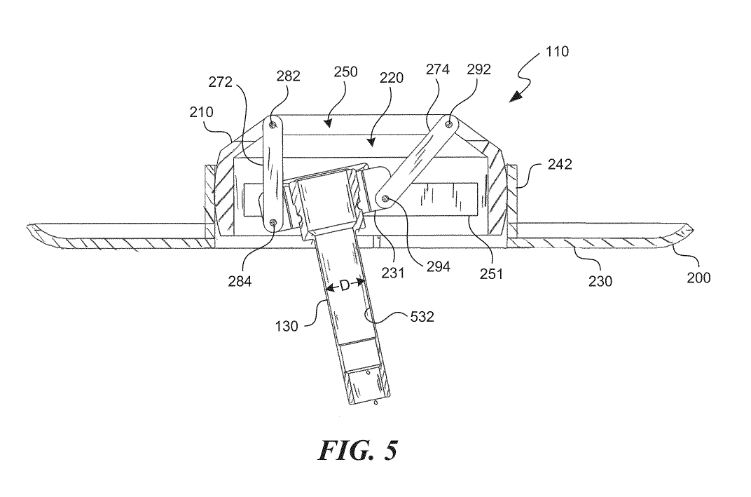

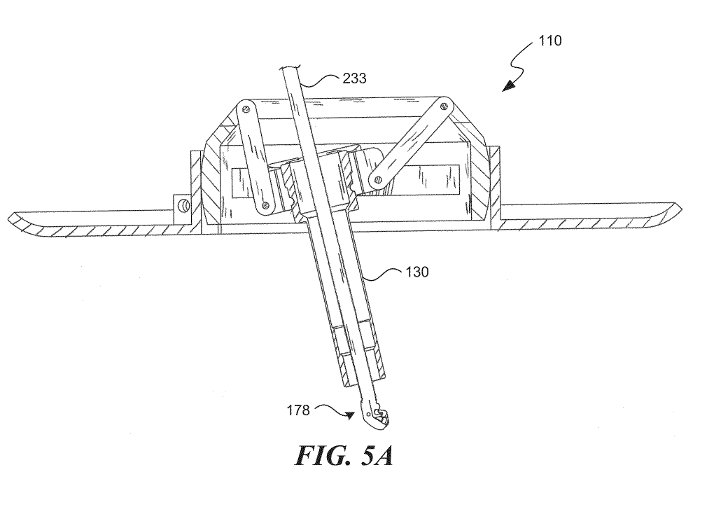

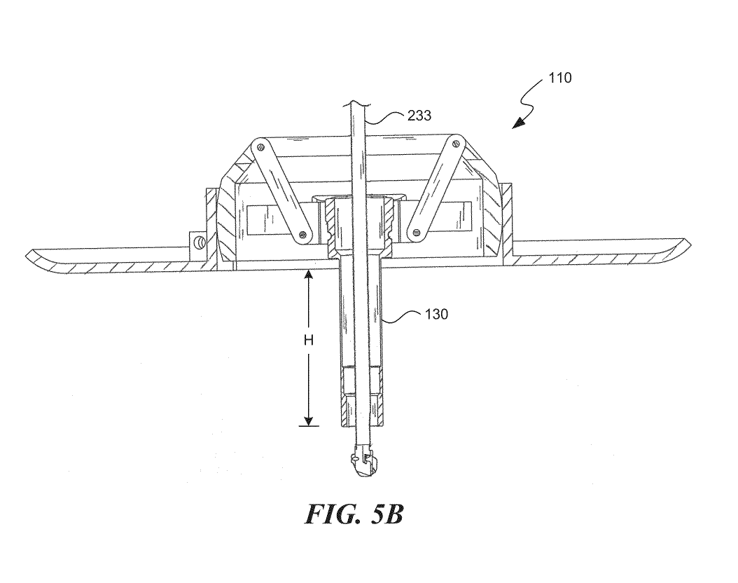

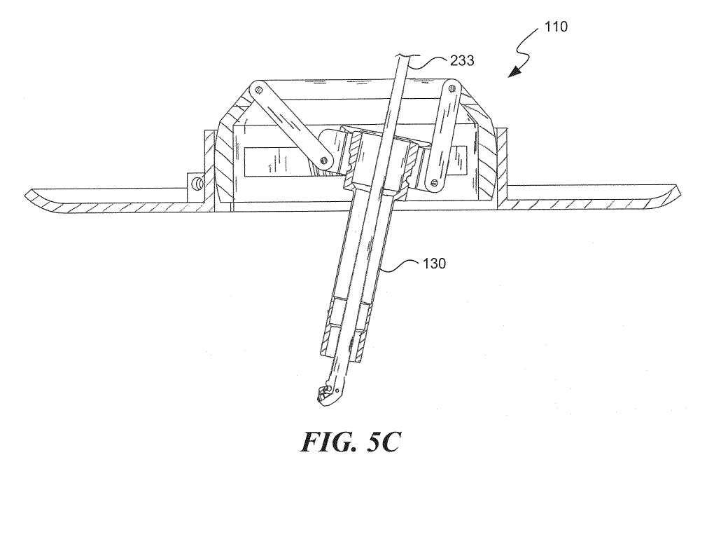

FIG. 3 is an isometric view, FIG. 4 is a top plan view, and FIG. 5 is a cross-sectional view of the positioner apparatus 110 in accordance with an embodiment of the disclosure. Referring to FIGS. 3-5 together, the positioner apparatus 110 includes a base 200, a holder 210, and a linkage assembly 220. The base 200 includes a plate region 230 and a retainer 242. The plate region 230 can be positioned on the patient or a spacer (e.g., a spacer 132 in FIG. 1). In some embodiments, the plate region 230 includes an adhesive or other feature for securing the plate region 230 to the patient's skin. In other embodiments, the plate region 230 can be placed directly on the patient.

Referring now to FIG. 5, the holder 210 is held by the retainer 242, and the linkage assembly 220 is coupled to the holder 210. The holder 210 can define an access opening 250 and includes a cross member 251. In some embodiments, the cross member 251 has one or more keying features, recesses, apertures, magnets, or the like that can receive or cooperate with a complementary feature of the linkage assembly 220. The linkage assembly 220 can include a collar 231 for holding the cannula 130 and links 272, 274 coupled to the collar 231. In one embodiment, the link 272 is rotatably coupled to the holder 210 and the collar 231 by pins 282 and 284, respectively. Similarly, a pin 292 rotatably couples the link 274 to the holder 210, and a pin 294 rotatably couples the link 274 to the collar 231. The links 272, 274 can rotate relative to the holder 210 and/or the collar 231 to move the cannula 130 between a first angled position (FIG. 5A), a centered position (FIG. 5B), and a second angled position (FIG. 5C) to provide lateral access and/or simultaneous bilateral access and to provide access the lamina, lateral recesses, facets (e.g., inferior facets), or the like. In FIGS. 5A-5C, a shaft 233 of the instrument 120 (FIG. 1) is positioned in the cannula 130.

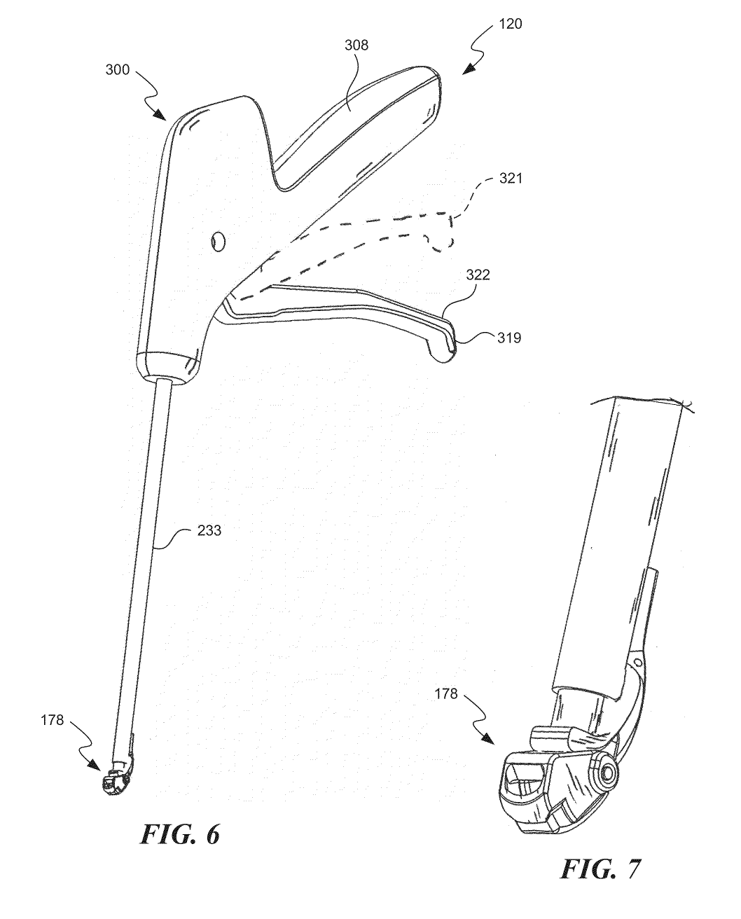

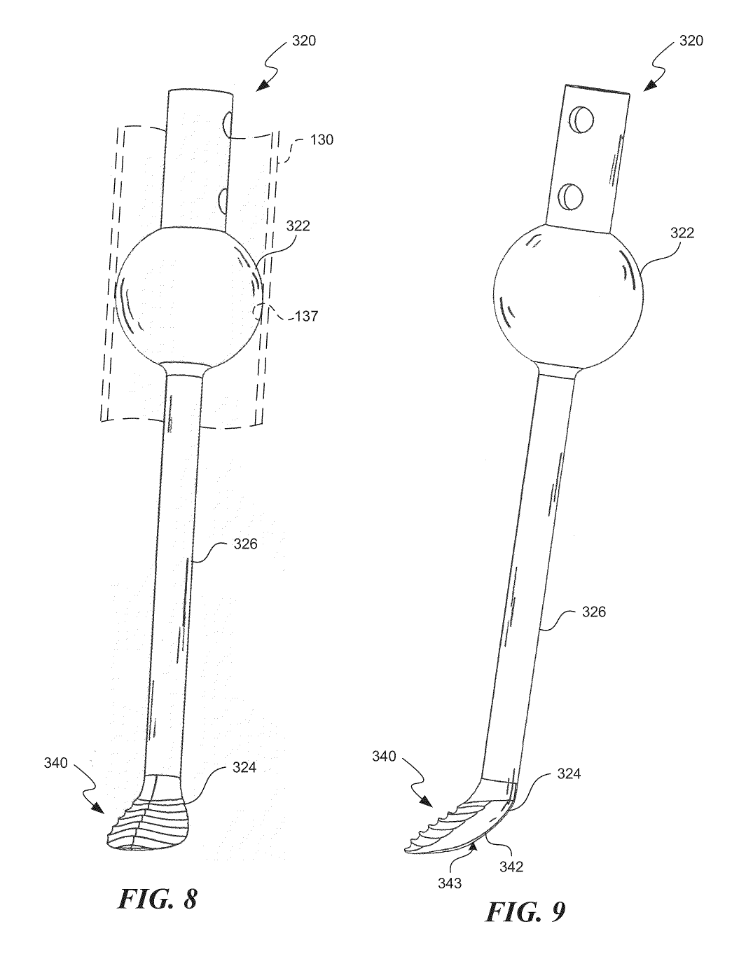

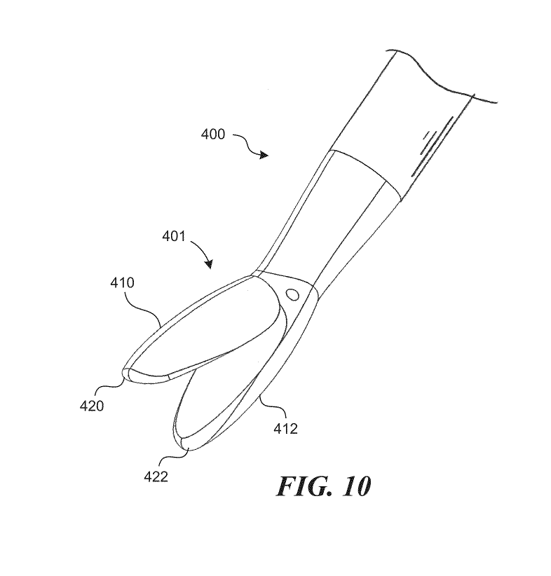

A surgical procedure can be performed using a series of instruments discussed in connection with FIGS. 6-10. FIGS. 6 and 7 show the tissue removal instrument 120. FIGS. 8 and 9 show a debulker instrument 320. FIG. 10 shows a tissue removal device 400 at a distal end of an instrument. Each of these instruments is discussed in detail below.

FIG. 6 is an isometric view of the instrument 120. FIG. 7 is an isometric view of the tissue removal device 178 of the instrument 120. Referring to FIG. 6, the instrument 120 includes an actuation mechanism 300 that can include a handle 308 and a lever 310 movable from a first position 319 (illustrated in dashed line) to a second position 321 to move the tissue removal device 178 from an open configuration (FIG. 2) to a closed configuration (FIGS. 6 and 7). The tissue removal device 178 can include a jaw that can be repeatedly moved between the open configuration and closed configuration to, for example, break, cut, scrape, crush, or otherwise alter tissue.

FIG. 8 is a front view of the debulker instrument 320 (“instrument 320”) configured in accordance with an embodiment of the disclosure. FIG. 9 is a side view of the instrument 320 of FIG. 8. The instrument 320 can include a positioning feature 322, a tissue altering tip 324, and an elongate shaft 326 extending between the positioning feature 322 and the tissue altering tip 324. The positioning feature 322 can allow rotation of the instrument 320 relative to the cannula 130 (a portion of the cannula 130 is shown in dashed line in FIG. 8). The positioning feature 322 can have a generally spherical shape or other suitable shape for rolling or pivoting relative an inner surface 137 of the cannula 130 to move the shaft 326. The tissue altering tip 324 can include, without limitation, one or more features (e.g., protrusions, grooves, blades, cutting edges, or the like), a textured surface, or other features for altering tissue. In some embodiments, including the embodiment of FIG. 9, the tissue altering tip 324 has an atraumatic portion 342 with a smooth curved surface 343 that can slide along tissue to inhibit, prevent, and substantially eliminate damage to tissue.

FIG. 10 is an isometric view of a tissue removal device 400 in accordance with an embodiment of the disclosure. The tissue removal device 400 includes a jaw assembly 401 with elongate jaws 410, 412 movable from an open configuration to a closed configuration to capture tissue. The elongate jaws 410, 412 can have atraumatic rounded or blunt tips 420, 422, respectively. Once tissue is captured in the jaw assembly 401, the jaw assembly 401 can be removed from the patient. The tissue removal device 400 can be used to clear the treatment site of loose tissue. The tissue removal device 400 can be connected to a wide range of different types of actuation mechanisms, including manually operated actuation mechanisms, such as the actuation mechanism 300 of FIG. 6.

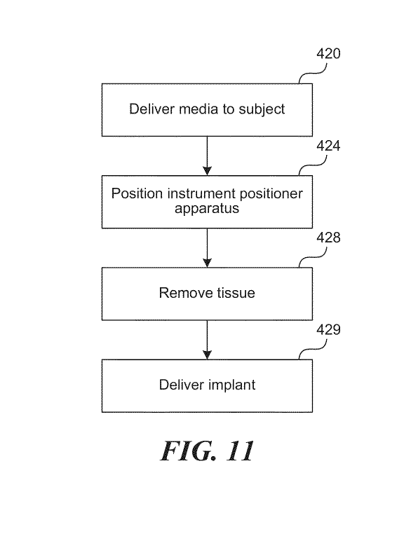

FIG. 11 is a flow chart of a method for reducing spinal nerve compression in accordance with an embodiment of the disclosure. At 420, media can be delivered to a media delivery site in the subject. The media can be visualization media suitable for viewing anatomical structures, tissue, and/or body fluids using fluoroscopy, magnetic resonance (MR) imaging, computer tomography (CT) imaging, or the like. The visualization media can include, without limitation, one or more contrast mediums, dyes (e.g., CT contrast agents), or the like. In MR imaging, the contrast medium can be a gadolinium based media.

The media delivery site can be in the dural sac, epidural space, space lateral to the spinal cord (e.g., space between or adjacent to motor roots), space with the spinal canal and adjacent to spinal ganglion), at the ligamentum flavum, or other suitable location. In some transforaminal injection procedures, visualization media can be delivered by, for example, positioning a needle of a delivery device against or proximate to the neural foramen. The delivery device can inject visualization media that travels into the spinal tissue, around the nerve roots, or the like. In some embodiments, the visualization media can travel throughout the epidural space without altering the tissue of the spinal cord.

In fluoroscopy imaging, the visualization media can be a radiopaque substance (e.g., a radiocontrast agent, a barium sulphate solution, etc.) or other substance for enhancing contrast of an image using radiography. In myelography procedures, the visualization media (e.g., a non-ionic contrast media) can be delivered directly into the spinal fluid surrounding the spinal cord via a media delivery device (e.g., a spinal needle) under fluoroscopy guidance. As such, the media can be kept outside of an epidural space (e.g., a portion 531 of the epidural space 533 in FIG. 13 between the spinal cord 182 and the ligamentum flavum 180). Myelography can provide detailed images (i.e., myelograms) of the spinal cord, thecal sac, nerve tissue (including nerve roots), or other features of interest. Additionally, myelography procedures can provide enhanced viewing of non-targeted structures (e.g., dura), nerve roots, etc. compared to epidurography procedures. For example, visualization media of a myelography procedure may travel (e.g., via controlled leakage) to nerve roots to visualize the nerve roots when removing bone of the neural foramen. In some embodiments, myelography visualization media can be used to verify decompression of the spinal cord because the dura can move outwardly to confirm that the pressure applied to the spinal cord is decreased or eliminated. If the dura is damaged (e.g., tears, leaks, or the like), myelography visualization media can escape out of the damaged region of the dura. A physician can view the leakage to confirm that the dura has been damaged, as well as identifying the location of the damage. The physician can then repair the dura or otherwise alter the surgical procedure. Accordingly, myelography visualization media can be used to provide useful real-time feedback.

At 424, the positioner apparatus 110 (FIG. 1) can be positioned on the patient. The cannula 130 (FIGS. 1 and 5) can be positioned within the patient, and an instrument can be delivered through the cannula 130.

At 428, the instrument can be used to remove targeted tissue. The targeted tissue can include, without limitation, bone (e.g., lamina, lateral recesses, facets including the inferior facets, etc.), bone spurs (e.g., bone spurs associated with osteoarthritis), tissue bulging from disks, tissue of thickened ligaments, spinal tumors, displaced tissue (e.g., tissue displaced by a spinal injury), or other tissue that may cause or contribute to spinal nerve compression. In procedures for treating stenosis, the instrument can be used to remove tissue associated with central canal stenosis, lateral recess stenosis, and/or other types of stenosis. The instrument can be viewed using fluoroscope, MR imaging, CT imaging, direct visualization, or the like.

At 429, additional procedures can be performed. In some embodiments, one or more devices can be implanted. The devices can be, for example, stabilizing devices, interspinous devices (e.g., interspinous spacers), or other suitable devices. Interspinous devices can be moved into interspinous spaces anteriorly through the cannula 130 (or other delivery conduit). In one procedure, a deployable interspinous device (e.g., an expandable interspinous spacer) can be deployed to engage and couple to the spinous processes or other features of vertebrae to, for example, reduce or limit spinal compression, pain, combinations thereof, or the like.

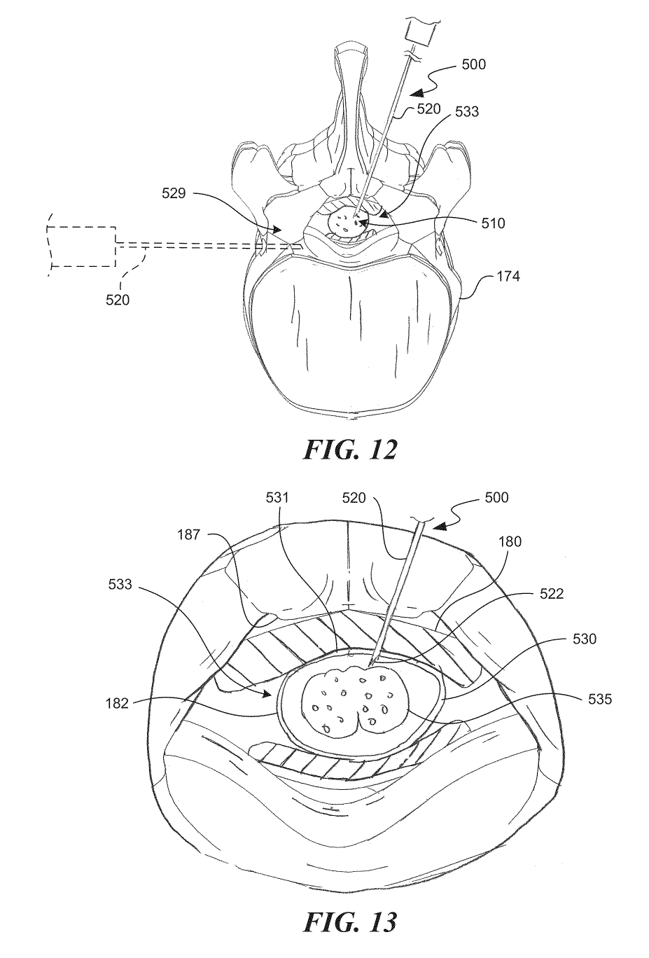

FIGS. 12-18 illustrate various stages of a spinal nerve decompression procedure in accordance with one embodiment of the disclosure. Referring now to FIG. 12, a media delivery device 500 (“delivery device 500”) is positioned to deliver visualization media to a media delivery site 510. The delivery device 500 can be a syringe or other suitable device for delivering visualization media. The size of the needle 520 can be, for example, a 22-26 gauge needle configured to pass between adjacent vertebrae. Needles having other different gauges can be used to prevent or limit headaches or other side effects. In some procedures, the positioner apparatus 110 is used for delivering the visualization media. The needle 520 can be moved through the cannula 130 which is then used to deliver and position surgical instruments.

FIG. 13 shows the delivery device 500 including a needle 520 (shown in solid line) with an outlet 522 positioned within a dural sac 530 to inject the visualization media into fluid surrounding the spinal nerves 535 (FIG. 12). The outlet 522 can be guided under fluoroscopy or other suitable imaging. Advantageously, substantially all the visualization media can be kept within the dural sac 530 to provide enhanced contrast of the margins of the dural sac 530. Additionally, visualization media can stay within the spinal cord 182 for an extended period of time without significant dispersion into other tissue in the vertebral foramen 187. In contrast, epidurography procedures can result in visualization media spreading throughout the spine.

In some embodiments, an epidurography procedure is performed. The needle 520 (shown in dashed line in FIG. 12) is positioned proximate to the neural foremen 529. The visualization media can be delivered by transforaminal injection to image tissue in the vertebral foramen 187. The media delivery site can also be at other locations as discussed in connection with FIG. 11.

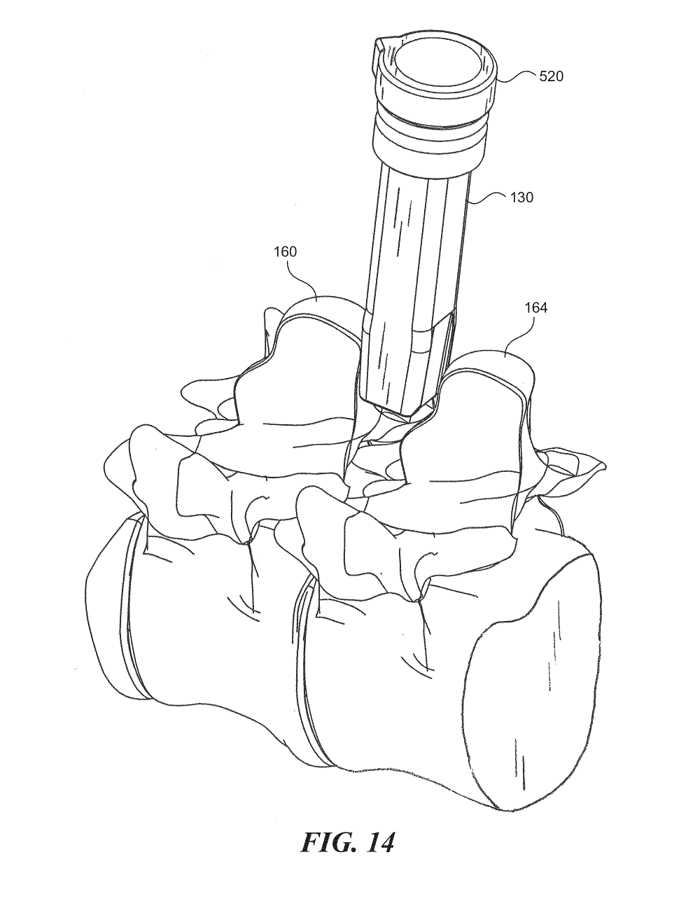

FIG. 14 is an isometric view of the cannula 130 positioned between the spinous processes 160, 164. For example, a posterior midline approach can be used to deliver the cannula 130 along a posterior-anterior direction to a location directly between the spinous processes 160, 164. In some embodiments, an incision is made in the supraspinal ligament 150 (FIG. 1) and the cannula 130 can be passed through the incision in the supraspinal ligament until it is inserted between the spinous processes 160, 164. Alternatively, ipsilateral or lateral approaches can be used to position the cannula 130.

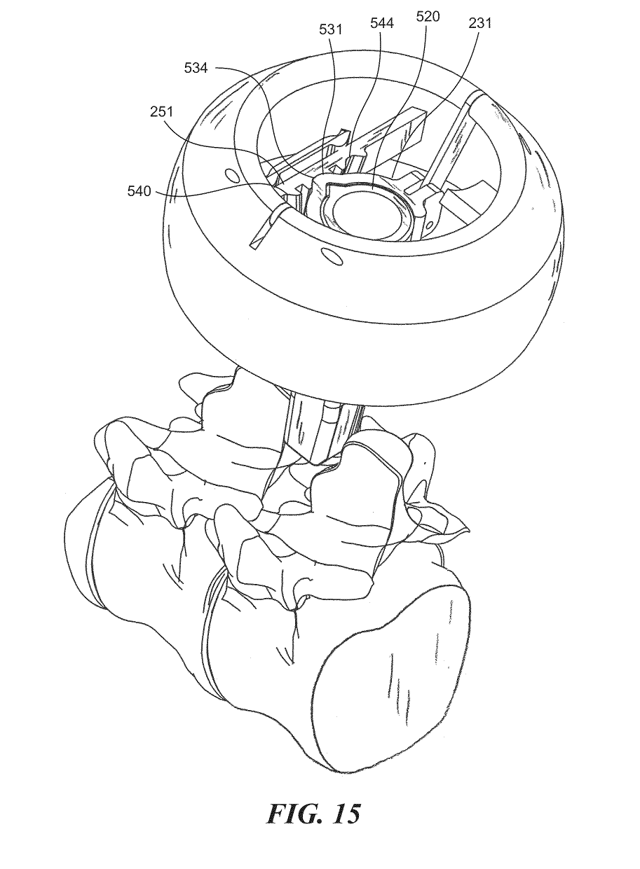

FIG. 15 is an isometric view of the partially assembled positioner apparatus 110. After positioning the cannula 130, the collar 231 can be coupled to a proximal end 520 of the cannula 130. A locator 531 of the collar 231 can be received by a recess 534 of the cross member 251. In the illustrated embodiment, the locator 531 can be locked at three discrete locking positions defined by recesses 540, 534, 544. FIGS. 5A-5C show the cannula 130 at the three corresponding positions. In other embodiments, the cross member 251 can define more or less than three locking positions.

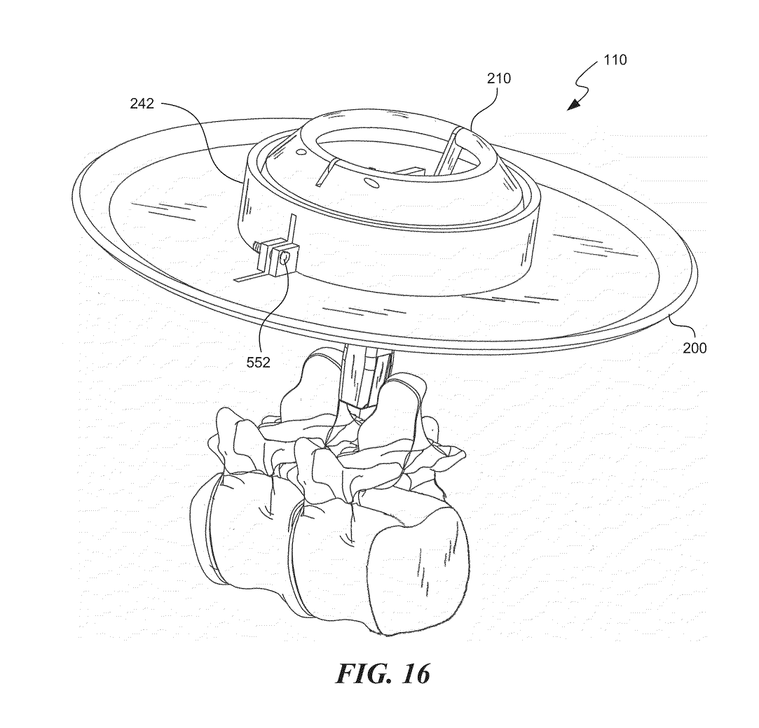

FIG. 16 is an isometric view of an assembled positioner apparatus 110 in accordance with an embodiment of the disclosure. The retainer 242 can be placed over the holder 210. The height H (FIG. 5B) can be increased or decreased by selecting the position of the retainer 242 relative to the holder 210. One or more fasteners 552 (e.g., screws, nut and bolt assemblies, or the like) can be used to tighten the retainer 242 about the holder 210. The assembled positioner apparatus 110 is ready to receive an instrument.

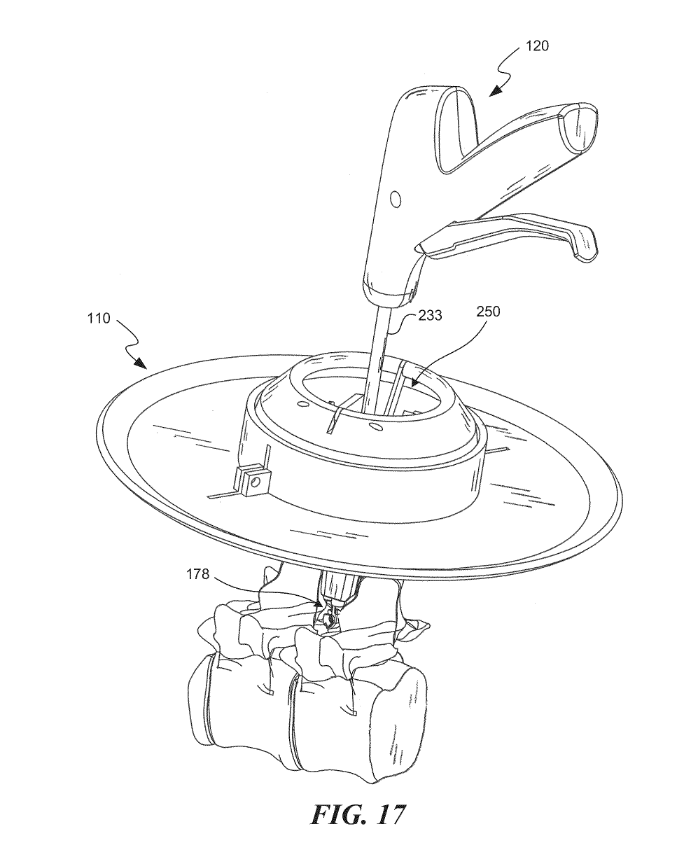

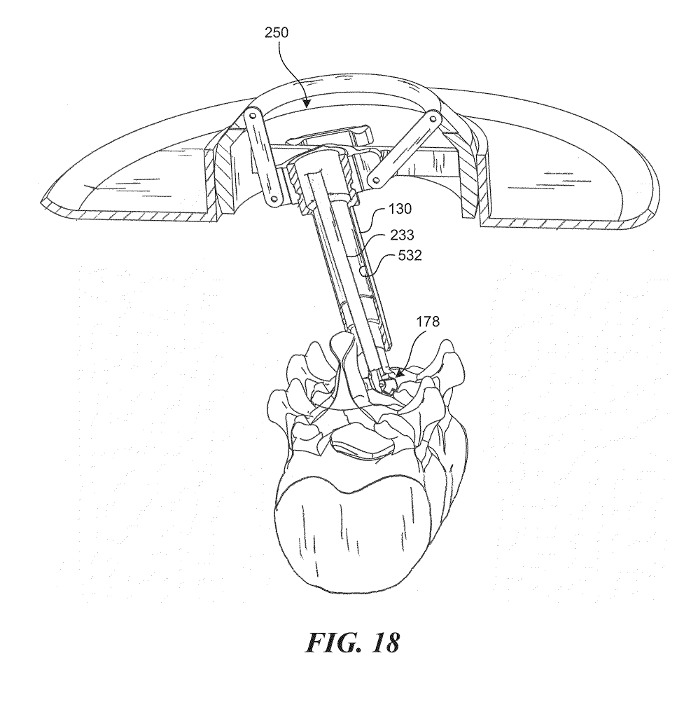

FIG. 17 is an isometric view of the assembled positioner apparatus 110 and the instrument 120. Referring now to FIGS. 1, 2, 17 and 18, the instrument 120 can be moved through the opening 250 and into a passageway 532 (FIG. 5) of the cannula 130 (shown in cross section in FIGS. 5 and 18). The tissue removal device 178 can be advanced through the passageway 532. The tissue removal device 178 of FIG. 18 can remove bone to, for example, reduce spinal compression, increase access to the treatment site, and can be viewed under fluoroscopy or other suitable visualization technique. The diameter D of the passageway 532 (FIG. 5) can be sufficiently large to allow repositioning of the instrument 120 to access different treatment sites, such as the lateral recesses, facets, ligamentum flavum, or the like. In some simultaneous bilateral access procedures, the cannula 130 can be repositioned while remaining in the patient to remove tissue from opposing lateral recesses or other lateral treatment sites. Additionally, the cannula 130 can provide direct visualization. For example, a user can view the treatment site and/or instrument by looking through the passageway 532 of the cannula 130. Additionally or alternatively, visualization devices (e.g., fiber optics, cameras, or the like) can be incorporated into the cannula 130 and/or instruments for viewing. After removing the desired amount of bone (or other tissue), the instrument 120 can be withdrawn from the subject.

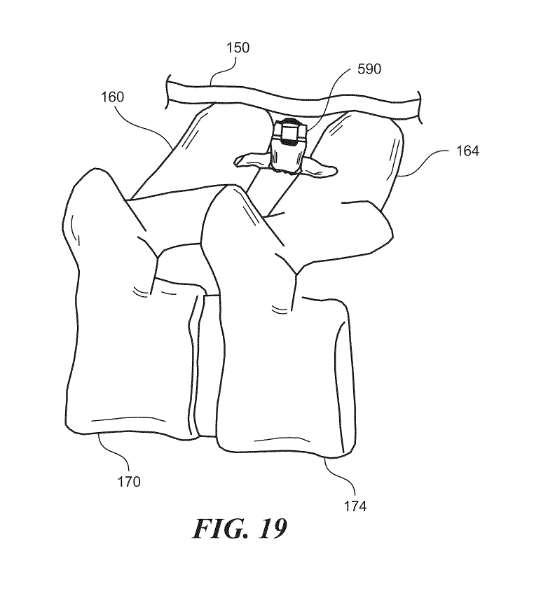

In some embodiments, the positioner apparatus 110 is used to deliver one or more spinal implants before, after, or during tissue removal. The methods of delivery, spinal implants, and other features of U.S. Pat. No. 8,012,207; U.S. Pat. No. 8,123,807; U.S. Pat. No. 8,152,837; and U.S. application Ser. No. 12/217,662 (corresponding U.S. Pub. No. 20080287997). U.S. Pat. No. 8,012,207; U.S. Pat. No. 8,123,807; U.S. Pat. No. 8,152,837; and U.S. application Ser. No. 12/217,662 (corresponding U.S. Pub. No. 20080287997) are hereby incorporated by reference in their entireties. FIG. 19 shows an implanted device 590 positioned between the spinous processes 160, 164. The device 590 can be delivered via the cannula 130 or other access device. In one embodiment, the device 590 is a SUPERION® Interspinous Spacer from VertiFlex, Inc. (San Clemente, Calif.) or a similar device. The device 590 can be implanted while imaging using visualization media and/or direct visualization.

Additional instruments can be utilized, including the instruments discussed in connection with FIGS. 8-10 and 20-25. The instrument 320 of FIGS. 8 and 9 can be used to separate the ligamentum flavum 180 from the lamina, cut tissue (e.g., the ligamentum flavum 180), debulk tissue (e.g., the ligamentum flavum 180), or combinations thereof. The tissue removal device 400 (FIG. 10) can be used to remove loose tissue (e.g., loose tissue from the ligamentum flavum 180). For example, tissue from different sections (e.g., sections within spinal foramina, sections between vertebrae, or the like) of the ligamentum flavum 180 can be removed. During a procedure, the cannula 130 can be moved between the three different positions discussed in connection with FIGS. 5A-5C.

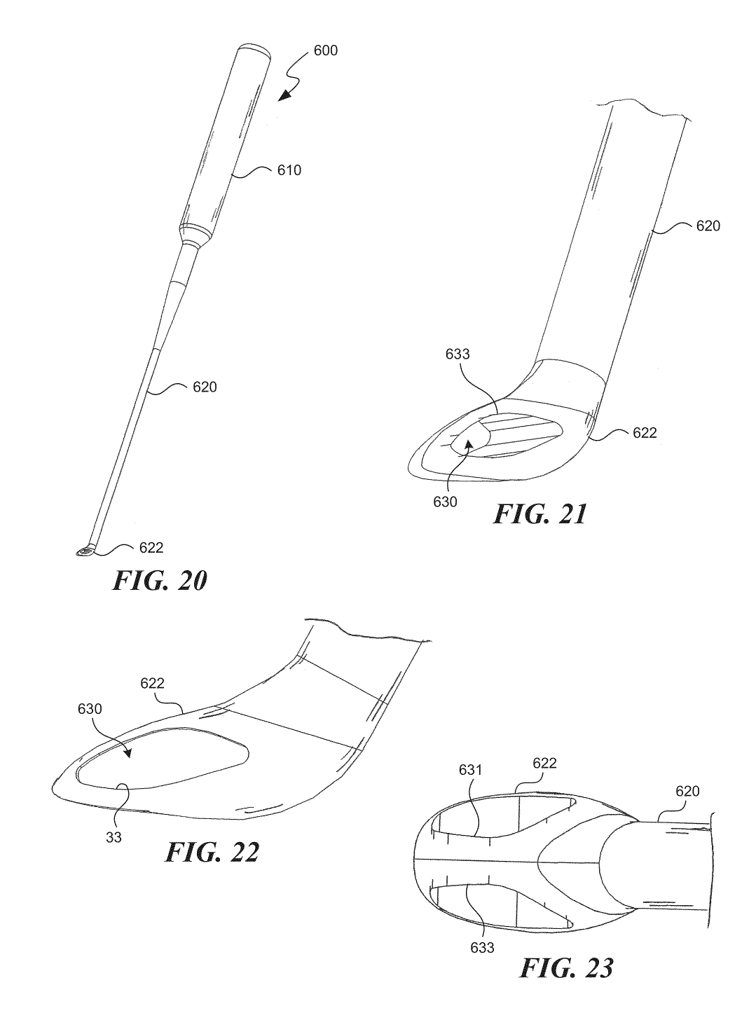

FIGS. 20-23 are various views of a debulker instrument 600 that includes a handle 610 (FIG. 20), an elongate shaft 620, and a debulking distal tip 622. The distal tip 622 has an opening 630 (FIG. 21) defined by cutting edges 631 (FIG. 23), 633 (FIGS. 22 and 23). The debulker instrument 600 can be manipulated within the subject to debulk the ligamentum flavum 180.

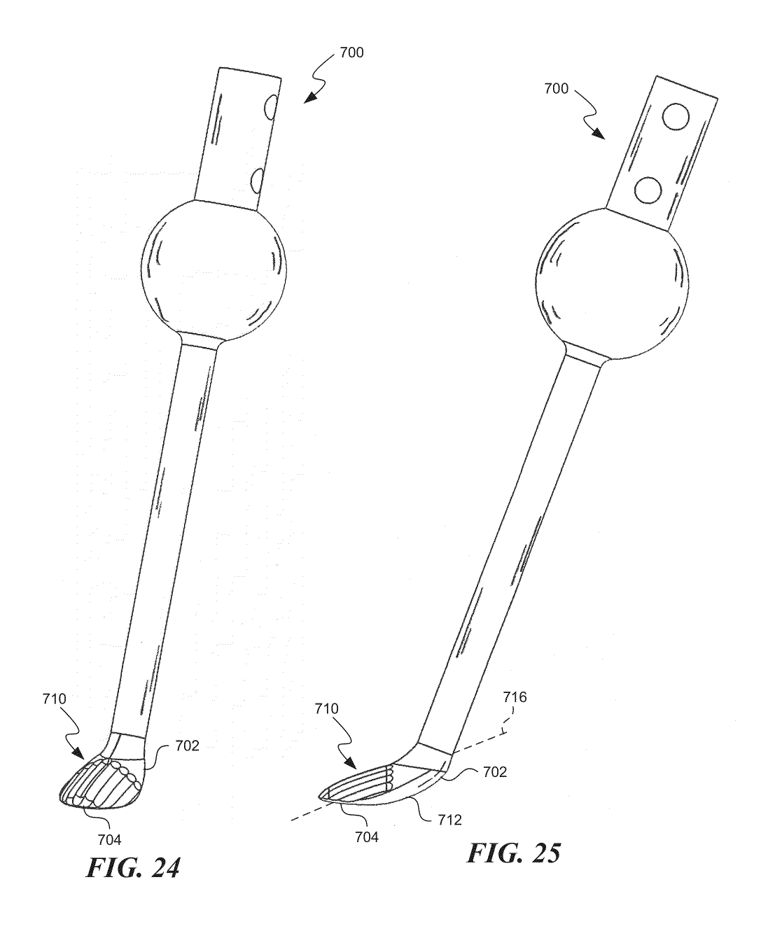

FIGS. 24 and 25 are front and side views of an instrument 700 in accordance with an embodiment of the disclosure. The instrument 700 includes a distal tip 702 with a head 704. The head 704 includes tissue altering features 710 extending longitudinally along the head 704. In some embodiments, the tissue altering features 710 can be grooves that extend generally parallel to a longitudinal access 716 of the head 704. The tissue altering features 710 can be used to scrape tissue, shave tissue, separate tissue(s), or the like.

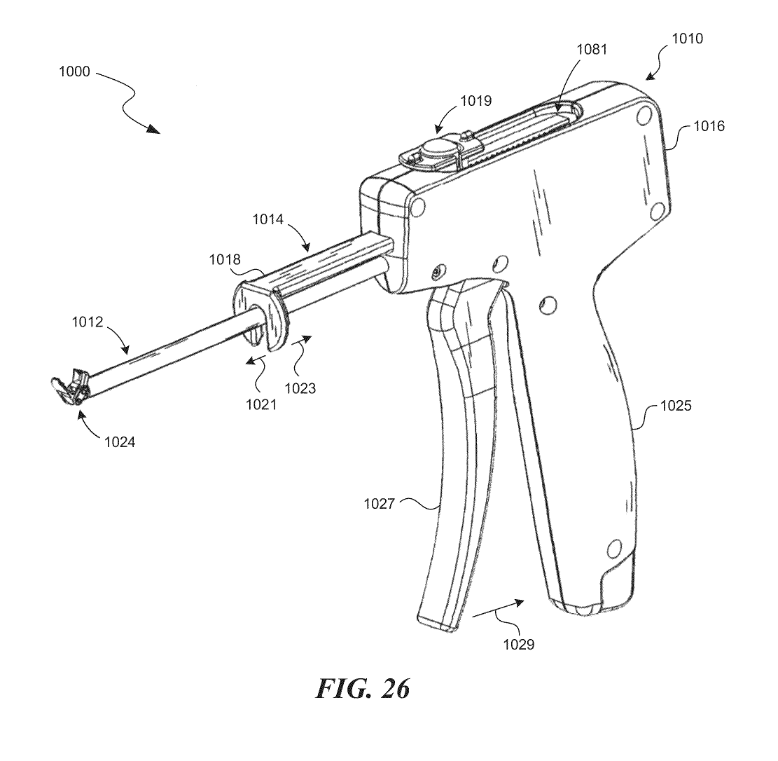

FIG. 26 is an isometric view of a tissue removal instrument 1000 (“instrument 1000”) in accordance with an embodiment of the disclosure. The instrument 1000 includes, without limitation, a tissue removal device 1012 and a holder in the form of an actuator mechanism 1010. The actuator mechanism 1010 includes, without limitation, a depth stop mechanism 1014 and a handle assembly 1016. The depth stop mechanism 1014 includes a stop member 1018 and a positioning assembly 1019. The positioning assembly 1019 can be used to move the stop member 1018 distally (indicated by arrow 1021) or proximally (indicated by arrow 1023) to adjust, for example, a maximum depth of penetration of the tissue removal device 1012. Once the stop member 1018 is at a desired location, the positioning assembly 1019 can be locked to hold the stop member 1018. The handle assembly 1016 includes a handle 1025 and a lever 1027. The handle 1025 can be manually held by a user and can be a pistol handle, a grip, or other suitable handhold. The lever 1027 can be pulled (indicated by arrow 1029) to close a jaw assembly 1024. Other types of handle assemblies can also be used.

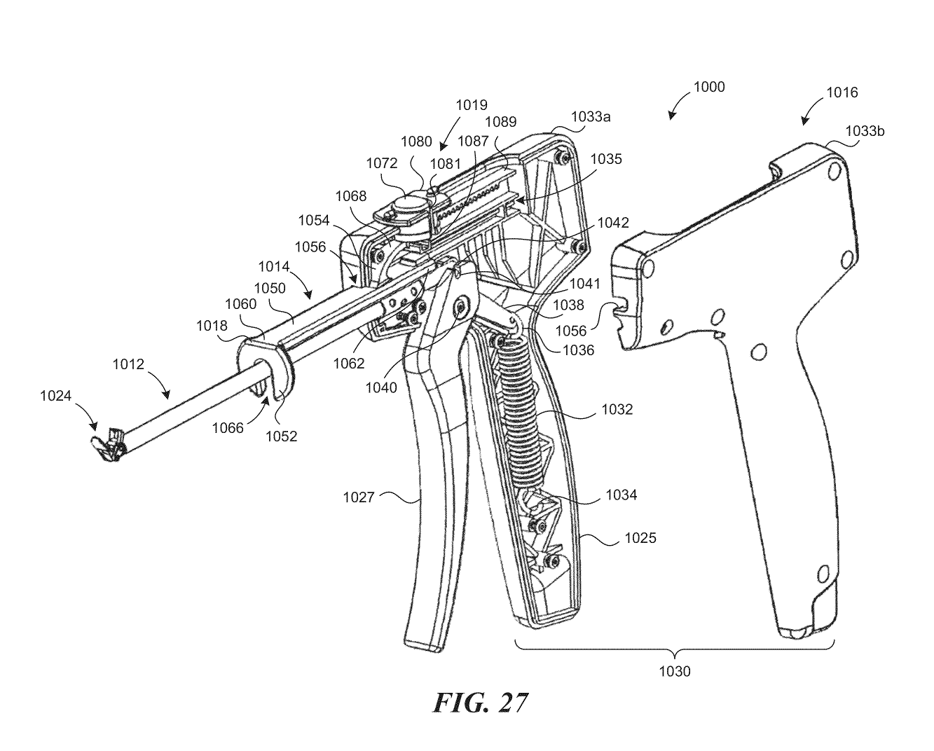

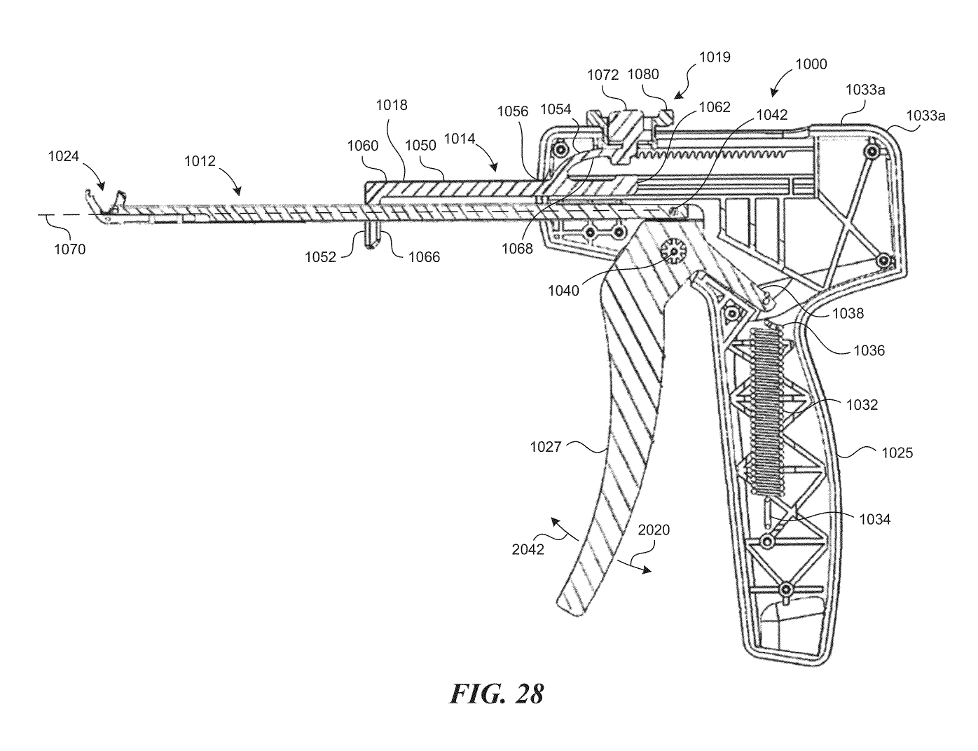

FIG. 27 is an isometric exploded view of the instrument 1000. FIG. 28 is a cross-sectional view of the instrument 1000. Referring to FIG. 27, the handle assembly 1016 includes a housing 1030 and a biasing device 1032. The housing 1030 can include housing portions 1033a, 1033b that surround and protect internal components. The housing portions 1033a, 1033b can include tracks 1035 (one identified in FIG. 27) along which the positioning assembly 1019 is capable of sliding. The biasing device 1032 can include a fixed end 1034 coupled to the housing portion 1033a and a mounting end 1036 coupled to an arm 1038 of the lever 1027. The biasing device 1032 can include, without limitation, a helical spring, an extension spring, or a coil spring and can be made, in whole or in part, of metal (e.g., spring steel, aluminum, etc.), plastic, or other material with desired mechanical properties to urge the lever 1027 to the illustrated initial position. A pin 1040 rotatably couples the lever 1027 to the housing portion 1033a. The lever 1027 can include a slot 1041 that receives a pin 1042 of the tissue removal device 1012. Other connections and components can be used to operably couple the actuator mechanism 1010 to the tissue removal device 1012.

FIG. 27 shows the stop member 1018 including a main body 1050, a head 1052, and a biasing element 1054. The main body 1050 extends through an opening 1056 in the housing 1030 and includes a distal end 1060 and a proximal end 1062. The head 1052 is coupled to the distal end 1060 and surrounds the tissue removal device 1012. In some embodiments, the head 1052 includes an opening 1066 in the form of a U-shaped slot. However, the opening 1066 can be a through hole or a slot having other configurations.

FIG. 28 shows the head 1052 generally perpendicular to the main body 1050 and/or a longitudinal axis 1070 of the tissue removal device 1012. However, the head 1052 can be at other orientations. The biasing element 1054 can include an arcuate member 1068 and a control element 1072. The arcuate member 1068 can be a flexure element that is integrally formed with or coupled to the main body 1050 and can be made, in whole or in part, of metal, plastic, or other materials with desired mechanical properties.

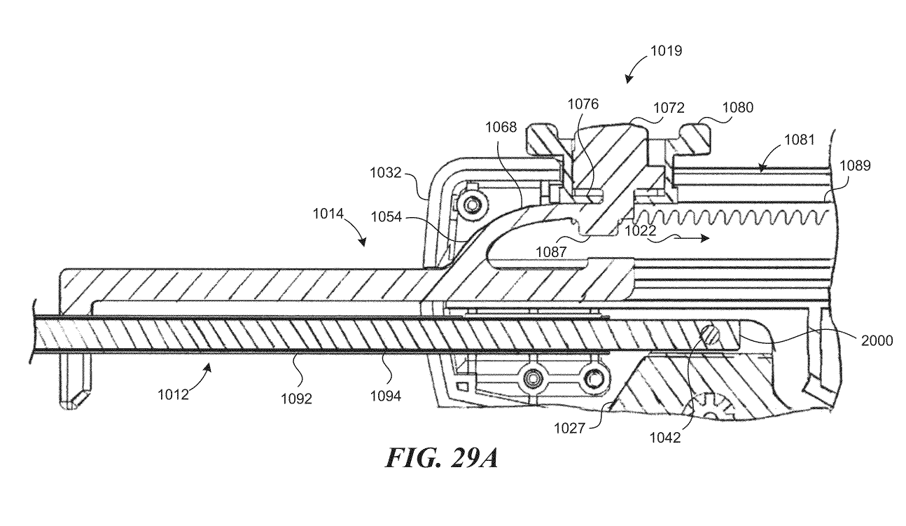

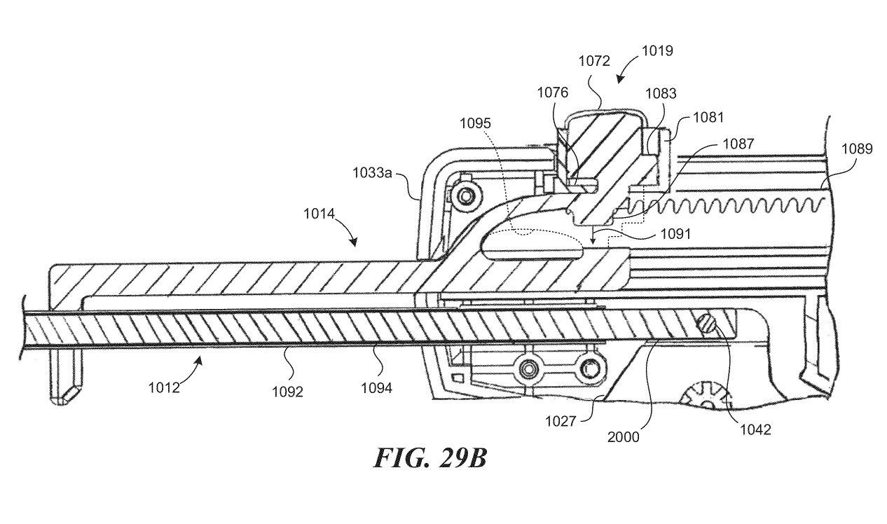

FIG. 29A is a detailed cross-sectional view of the depth stop mechanism 1014 in a locked configuration. FIG. 29B is a detailed cross-sectional view of the depth stop mechanism 1014 in an unlocked configuration. The positioning assembly 1019 can include a control element 1072, a locking element 1080, and a ratchet 1089. The control element 1072 can be a button or lever and can be movable from a first position (e.g., an undepressed position, an extended position, etc.) to a second position (e.g., a depressed position, an unextended position, etc.). A plate 1076 of the locking element 1080 keeps the control element 1072 in the first position such that an engagement member 1087 can enmesh or otherwise engage one or more features (e.g., teeth, notches, etc.) of the ratchet 1089. The locking element 1080 has a slot 1081 and can be rotated from a located position (FIG. 27) to an unlocked position (FIG. 29B).

To move the positioning assembly 1019 from a locked configuration (FIG. 29A) to an unlocked configuration (shown in dashed line in FIG. 29B), the locking element 1080 is rotated to align the slot 1081 with a protrusion 1083 of the control element 1072. A user can press down on the control element 1072 to overcome a biasing force provided by the biasing element 1054 to move the control element 1072 (indicated by arrow 1091) to disengage the engagement member 1087 from the ratchet 1089. After moving the control element 1072 to a depressed position 1095 (illustrated dashed line in FIG. 29B), the control element 1072 can be moved proximally or distally. After the depth stop mechanism 1014 is moved to a desired position, the control element 1072 can be released. The biasing element 1054 can move the control element 1072 to the undepressed position. The locking element 1080 can be rotated to the locked position.

Other types of depth stop mechanisms can be used and can include, without limitation, one or more biasing devices (e.g., springs, actuators, etc.), control elements, or the like. The configuration and functionality of the depth stop mechanism can be selected based on the desired operation of the instrument 1000.

FIG. 29B shows the tissue removal device 1012 including, without limitation, an outer member 1092 and an inner member 1094. The outer member 1092 is fixedly coupled to the housing portion 1033a. The inner member 1094 includes a proximal end 2000 coupled to the lever 1027 via the pin 1042. In some embodiments, the inner member 1094 comprises a cylindrical push rod (e.g., a solid push rod, a hollow push rod, etc.). The tissue removal device 1012 can have other components, arrangements, and configurations.

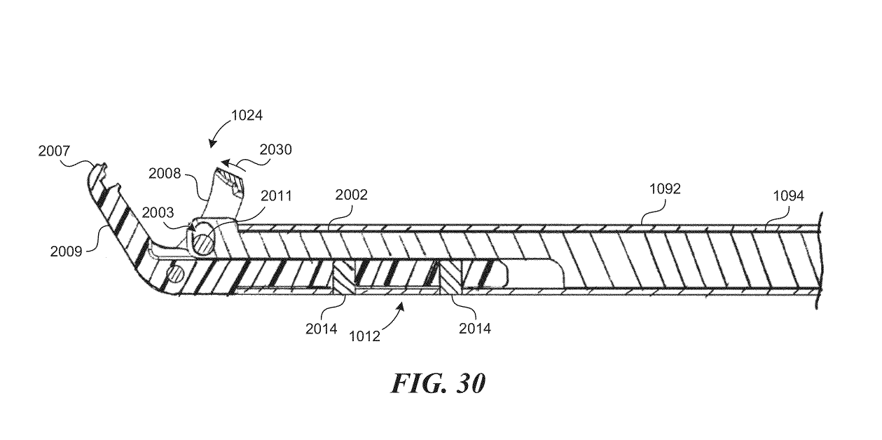

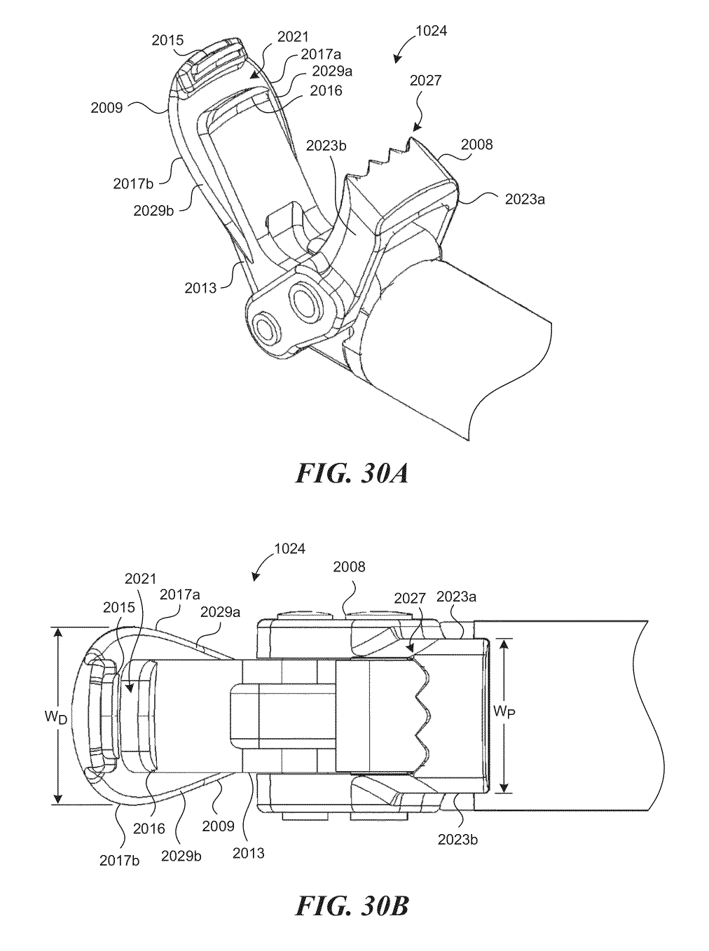

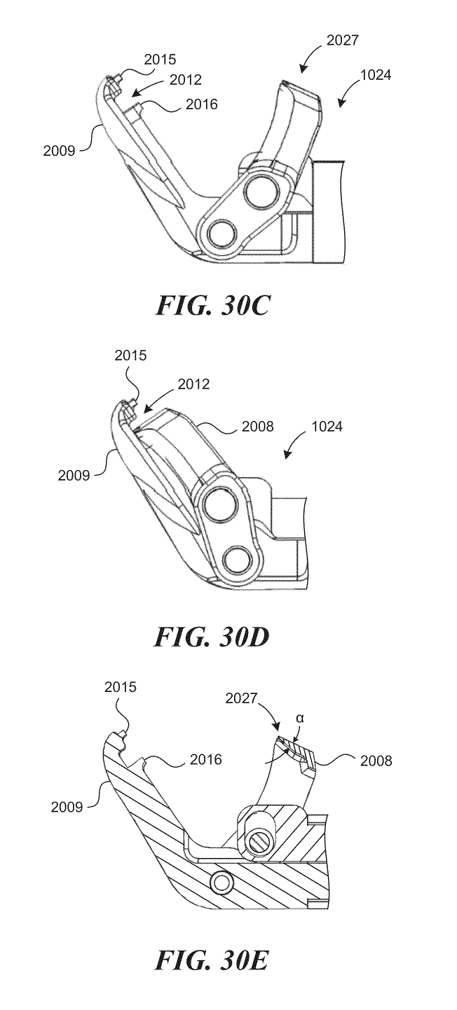

FIG. 30 is a detailed cross-sectional view of the jaw assembly 1024 that includes proximal and distal jaws 2008, 2009. The jaw 2008 is coupled to a distal end 2002 of the inner member 1094 via a pin 2011 in an opening 2003. In some embodiments, the jaw 2009 has an atraumatic configuration and includes a rounded or curved surface 2007 configured to slide along tissue without, for example, damaging or traumatizing the tissue. The jaw 2009 is fixedly coupled to the outer member 1092 via one or more fasteners 2014 (e.g., pins, screws, etc.). When the lever 1027 (FIG. 28) is rotated about the pin 1040, the lever 1027 pushes the inner member 1094 distally through the outer member 1092 to cause rotation of the jaw 2008 (indicated by arrow 2030 of FIG. 30), as the inner member 1094 moves distally. To open the closed jaw assembly 1024, the user can release the lever 1027. The biasing device 1032 can pull the lever 1027 about the pin 1040 (indicated by an arrow 2042 in FIG. 28). The lever 1027 pulls the inner member 1094 proximally to move the jaw 2008 to the open position. The lever 1027 can be used to repeatedly open and close the jaw assembly 1024.

FIG. 30A is an isometric view of the jaw assembly 1024. FIG. 30B is a top view of the jaw assembly 1024. Referring to FIGS. 30A and 30B together, the distal jaw 2009 can include a base 2013 and a pair of protrusions 2015, 2016. A receiving channel 2021 is defined by the protrusions 2015, 2016 and can be a U-shaped channel, a V-shaped channel, or other type of channel configured to receive cutting features 2027 of the jaw 2008. The base 2013 includes lateral or edge portions 2017a, 2017b that extend outwardly past the respective sides 2023a, 2023a of the jaw 2008 sufficient distances to keep tissue away from cutting features 2027. A ratio of the width W_{D} of the jaw 2009 to the width Wₚ of the jaw 2008 can be equal to or greater than about 1.1, 1.2, and 1.4. As such, the lateral portions 2017a, 2017b can serve as protective guards. Other ratios are also possible. In some embodiments, the lateral portions 2017a, 2107b have atraumatic edges 2029a, 2029b, respectively, for sliding along tissue. In other embodiments, the edges 2029a, 2029b can be sharp to cut tissue.

FIG. 30C is a side view of the open jaw assembly 1024. The protrusion 2015 can be used to provide tactile feedback to the user. For example, the protrusion 2015 can be used to contact tissue to determine the location of the jaw assembly 1024. In some embodiments, the protrusion 2015 includes an atraumatic tooth. In other embodiments, the protrusion 2015 includes a plurality of cutting teeth. The protrusions 2015, 2016 and the cutting features 2027 can cooperate to, for example, break, crush, cut, or otherwise facilitate removal of material from the subject. Referring to FIG. 30D, the cutting features 2027 can be moved into the receiving channel 2021. The material can be contained in the closed jaw assembly 1024 for convenient removal from the subject.

FIG. 30E is a cross-sectional view of the open jaw assembly 1024. The cutting features 2027 can define an included angle α that is in a range of about 5 degrees to about 60 degrees. In some embodiments, the angle α is a range of about 20 degrees to about 50 degrees to help dig into material (e.g., bone, ligament tissue, etc.). Other angles α can also be used to achieve the desired cutting action.

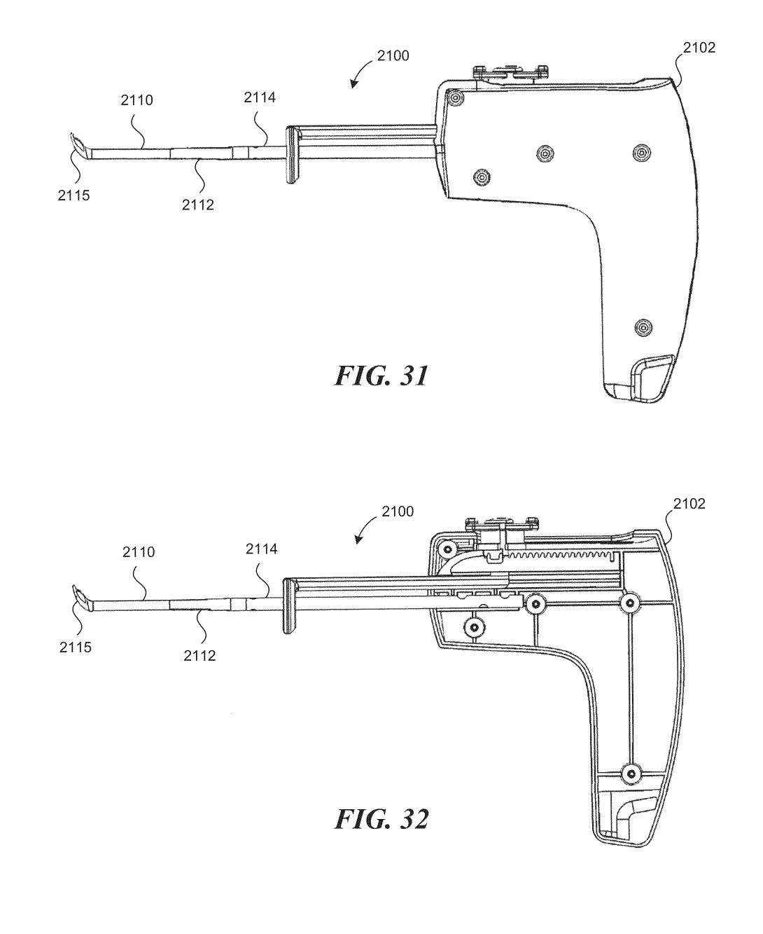

FIG. 31 is a side view of a debulker instrument 2100 (“instrument 2100”) in accordance with an embodiment of the disclosure. FIG. 32 is a side view of internal components of the instrument 2100. The instrument 2100 is generally similar to the instrument 1000 of FIGS. 26-30E, except as detailed below. The instrument 2100 of FIGS. 31 and 32 includes a handle assembly 2102 and a tool 2110 fixedly coupled to the handle assembly 2102. The tool 2110 includes a main body 2112 and a distal portion 2115. The main body 2112 is removably coupled to a tool holder 2114 (“holder 2114”) of the handle assembly 2102.

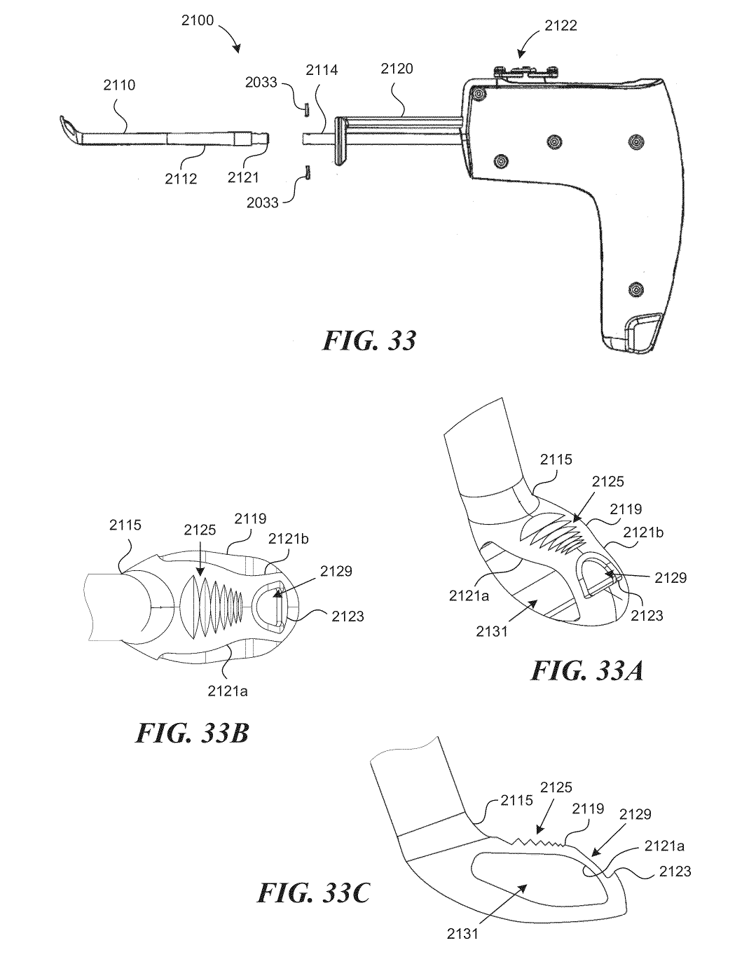

FIG. 33 is a side view of the instrument 2100 with the tool 2110 ready to be installed in the holder 2114. Fasteners 2030 (e.g., pins, screws, etc.) can be used to couple the tool 2110 to the holder 2114. The holder 2114 extends through a head 2118 of a stop member 2120. A locking mechanism 2122 of a depth stop mechanism can be unlocked to move the stop member 2120 axially along the holder 2115. The fasteners 2030 can be removed to replace the tool 2110 with another tool (e.g., a reaming tool, visualization instrument, a cutter, jaw assembly, etc.).

FIG. 33A is an isometric view of the distal portion 2115. FIG. 33B is a top view of the distal portion 2115. FIG. 33C is a side view of the distal portion 2115. The distal portion 2115 includes a debulking head 2119 with cutting edges 2121a, 2121b, a distal engagement region 2123, and a proximal engagement region 2125. The engagement regions 2123, 2125 can include, without limitation, texturing, cutting edges, protrusions, openings (e.g., access openings) or other features capable of loosening, separating, cutting, scraping, or otherwise effecting or receiving tissue. The number, positions, and configurations of the engagement regions can be selected based on the procedure to be performed. In some procedures, loose tissue can pass through an access opening 2129 and can collect in a chamber 2131 (FIGS. 33A and 33C). The debulking head 2119 can be removed from subject and the chamber 2131 can be emptied.

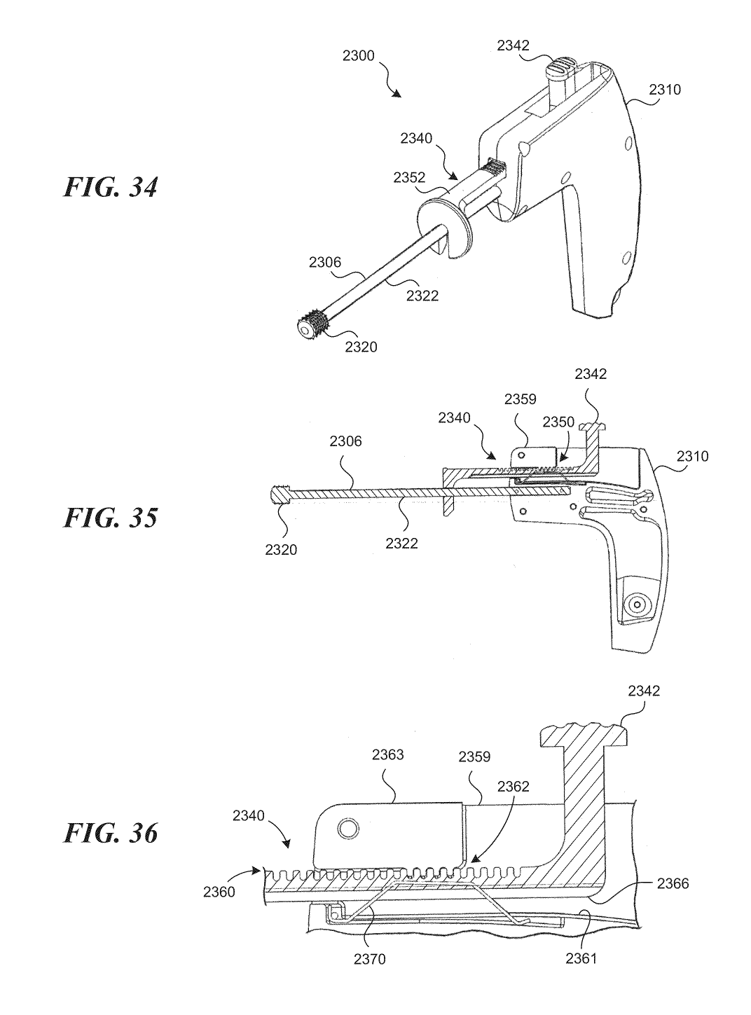

FIG. 34 is an isometric view of a reamer instrument 2300 (“reamer instrument 2300”) in accordance with an embodiment of the disclosure. FIG. 35 is a cross-sectional view of the instrument 2300. FIG. 36 is a detailed cross-sectional view of a depth stop mechanism 2340 of the instrument 2300. Referring to FIGS. 34 and 35 together, the instrument 2300 includes, without limitation, a tool 2306 and a handle assembly 2310. The tool 2306 can include a shaft 2322 and a distal portion in the form of a head 2320. The head 2320 can be a reamer head (e.g., a head with a textured surface, a plurality of protrusions, etc.) that is configured to abrade, scrape, or otherwise alter tissue.

The depth stop mechanism 2340 of FIG. 35 has a locking assembly 2350 and a stop member 2344. Referring to FIG. 36, the locking assembly 2350 includes, without limitation, a control element 2352 and a portion 2363 of a housing 2359. In some embodiments, engagement features 2360 of the control element engage engagement features 2362 of the portion 2363. The engagement features 2360, 2362 can be teeth, grooves, or the like. The locking assembly 2350 can be movable from a locked position (FIGS. 35 and 36) to an unlocked position in which a surface 2361 of the housing 2359 contacts a surface 2367. A biasing device 2370 can urge the locking assembly 2350 towards the locked configuration and can include, without limitation, one or more angled members (one angled member is illustrated), springs (e.g., helical springs), or the like. A user can press down on the control element 2342 to disengage the engagement features 2360 from the engagement features 2362.

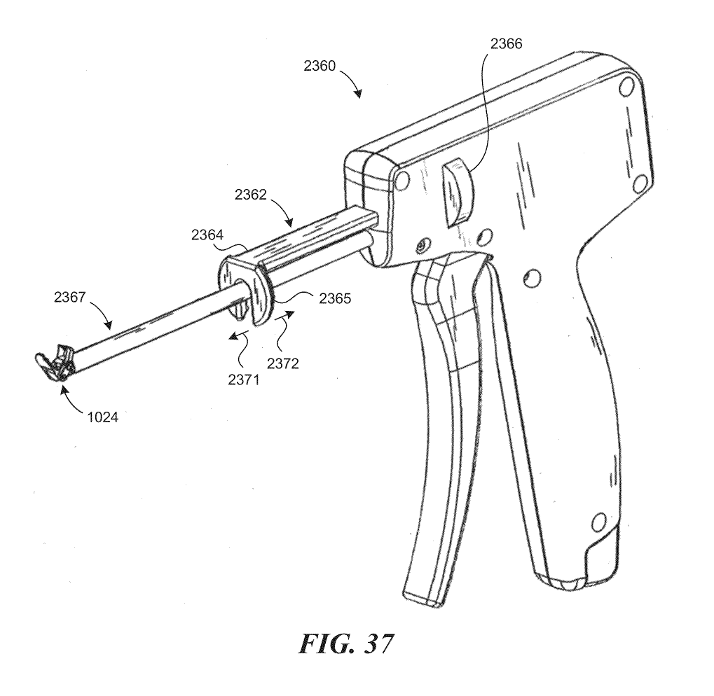

FIG. 37 is an isometric view of a tissue removal instrument 2360 (“instrument 2360”) in accordance with an embodiment of the disclosure. The instrument 2360 includes, without limitation, a depth stop mechanism 2362 including a stop member 2364 and an adjuster 2366. A user can rotate the adjuster 2366 to move a head 2365 of the stop member 2364 along a tool 2367. Other types of drive components or mechanisms can also be used. The adjuster 2366 can include, without limitation, threaded members, drive components, or the like that cause movement of the stop member 2364 in the distal direction (indicated by arrow 2371) and in the proximal direction (indicated by arrow 2372).

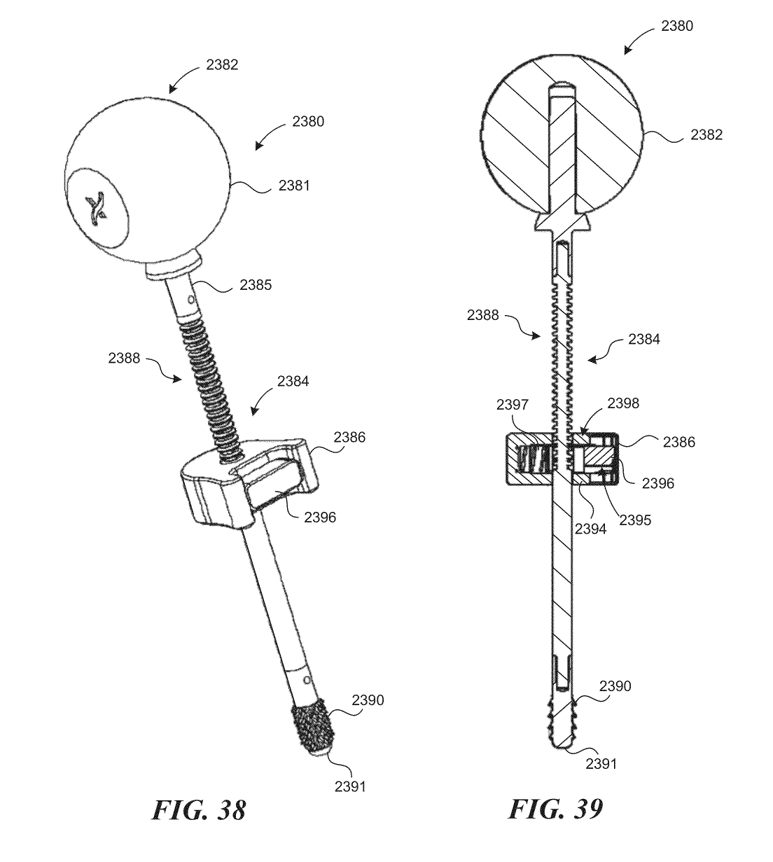

FIG. 38 is an isometric view of an instrument 2380 in accordance with an embodiment of the disclosure. The instrument 2380 can include, without limitation, a handle assembly 2382 and a remear tool 2384. The handle assembly 2382 includes a handle 2381 and a tool holder 2385. The remear tool 2384 can include a depth stop mechanism 2386 (“stop mechanism 2386”), a ratchet portion 2388, and a reamer head 2390. The reamer head 2390 includes an atraumatic tip 2391 (e.g., a rounded tip, a blunted tip, etc.). The stop mechanism 2386 is coupleable to the ratchet portion 2388 to adjust the depth of penetration. In some embodiments, the stop mechanism 2386 can allow the user to adjust the maximum depth of penetration of the reamer head 2390 at desired increments (e.g., 1 mm increments, 2 mm increments, 4 mm increments, etc.).

Referring to FIG. 39, the stop mechanism 2386 can include a stop member 2394 and a locking assembly 2395. The locking assembly 2395 has a locked configuration for holding the stop member 2394 against the ratchet portion 2388 and an unlocked configuration for moving the stop member 2394. In some embodiments, the locking assembly 2395 can include a control element 2396, a biasing device 2397, and an engagement member 2398. The control element 2396 can include, without limitation, one or more buttons. The biasing device 2397 can keep features (e.g., teeth) of the engagement member 2398 in contact with features (e.g., teeth, annular members, grooves, etc.) of the ratchet portion 2388. A user can press on the control element 2396 to overcome the biasing device 2397 to disengage the engagement member 2398 and the ratchet portion 2388. Once the locking assembly 2395 is in the unlocked configuration, the stop mechanism 2386 can be moved axially along ratchet portion 2388.

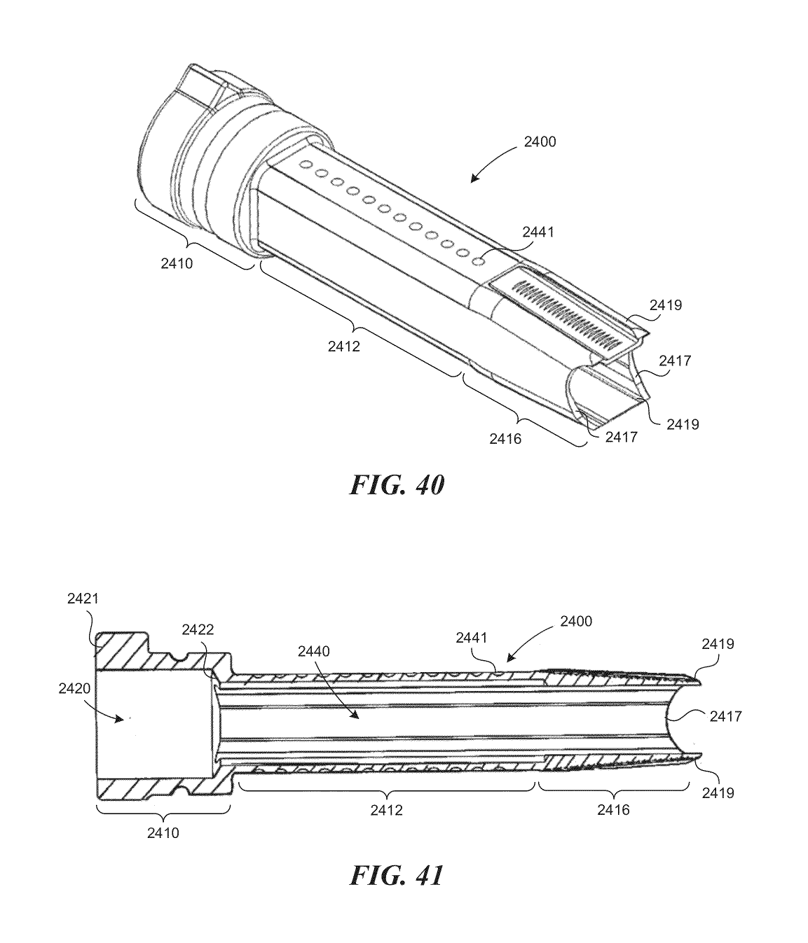

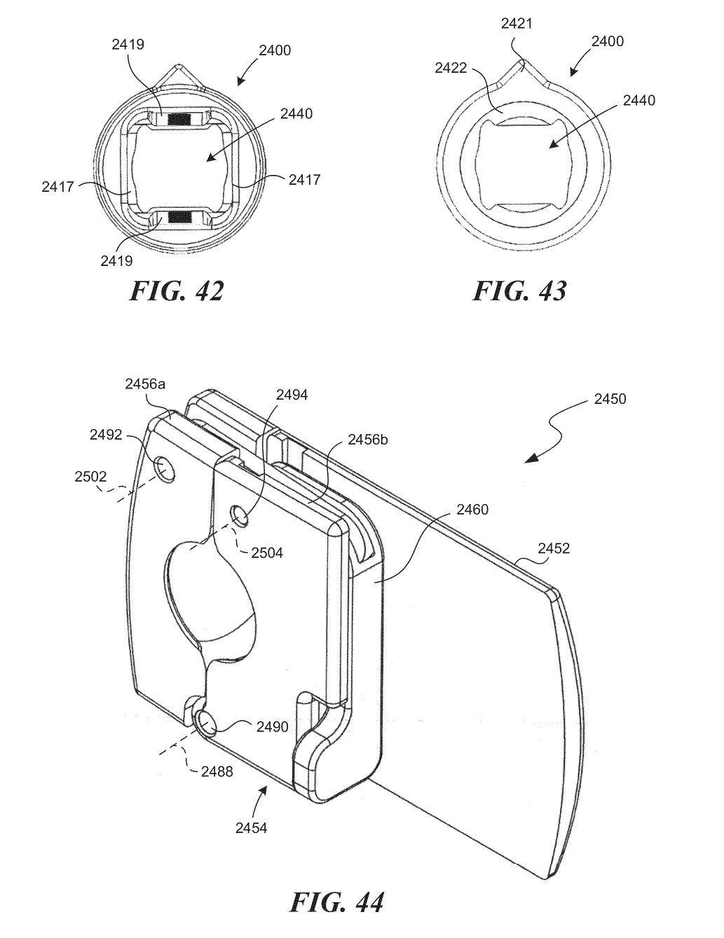

FIG. 40 is an isometric view of a cannula 2400 in accordance with an embodiment of the disclosure. FIG. 41 is a cross-sectional view of the cannula 2400. FIGS. 42 and 43 are end views of the cannula 2400. Referring to FIGS. 40 and 41 together, the cannula 2400 includes a head 2410, a main body 2412, and a distal end 2416. The head 2410 defines a receiving opening 2420, a surface 2421, and a shoulder 2422. The surface 2421 and/or shoulder 2422 can serve as a stop. When a tool is positioned in a passageway 2440, a head of a depth stop mechanism can contact the surface 2421 and/or shoulder 2422.

The main body 2412 includes keying features 2441 (one of twelve positioning features is identified). The illustrated cannula 2400 has a generally straight array of spaced apart keying features 2441 in the form of partially spherical recesses, but a greater or lesser number of keying features can be selected based on the desired number of locking positions for a collar, the length of the cannula 2400, etc. As shown in FIG. 41, the keying features 2441 can be located on opposing sides of the main body 2412. Other types of keying features in the form of elongated recesses, dimples, protrusions (e.g., partially spherical protrusions, elongated protrusions, etc.), or other discrete features can be used.

The distal end 2416 can be configured to be positioned in the subject proximate to the treatment site. For example, the distal end 2416 can be positioned between adjacent vertebrae or at another desired site. In some embodiments, relief features 2417 increase access to lateral regions of the patient and can be cut-outs or other features that increase accessibility of lateral regions while shielding portions 2419 are positioned adjacent to, for example, spinous processes. Other types of cannulas or delivery instruments having other configurations and features can also be used.

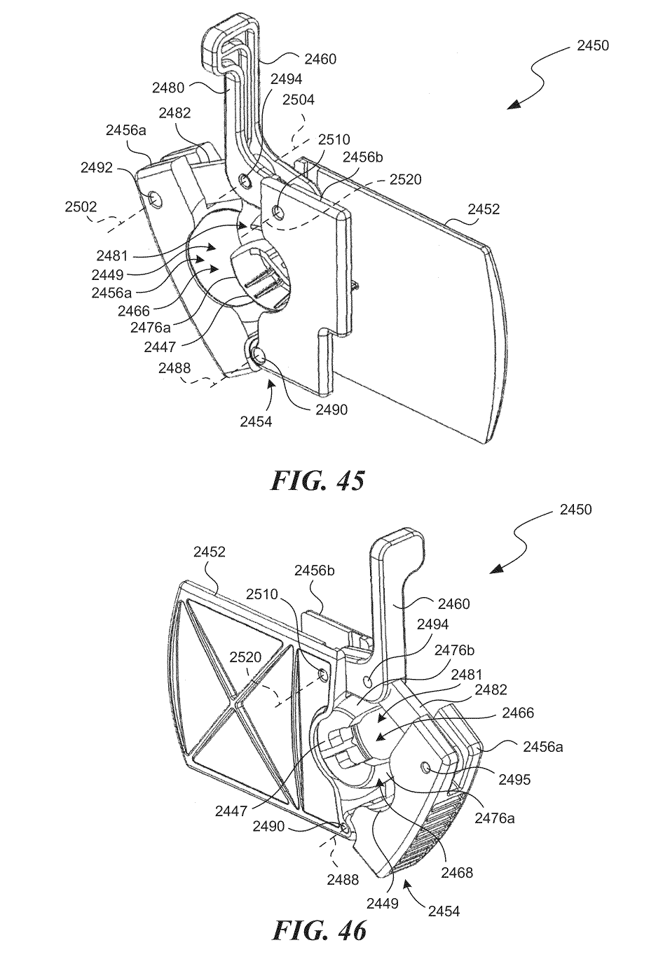

FIGS. 44 and 45 are isometric views of an instrument positioner apparatus 2450 in accordance with an embodiment of the disclosure. Referring to FIG. 44, the instrument positioner apparatus 2450 includes, without limitation, a base 2452 and a clamp assembly 2454. The base 2452 can be a rigid plate carrying the clamp assembly 2454. The clamp assembly 2454 can include jaws 2456a, 2456b and a latch mechanism 2460 movable between a closed configuration (FIG. 44) and an open configuration (FIGS. 45-47).

FIG. 48 shows the clamp assembly 2454 including a joint device 2466 including a collar 2447 rotatably relative to a socket 2449 defined by the jaws 2456a, 2456b. In some embodiments, the collar 2447 has a surface 2468 (e.g., a curved surface, a partially spherical surface, etc.) that can slideably engage complementary surfaces 2470a, 2470b (e.g., curved surfaces, partially spherical surfaces, etc.) of the socket 2449. When the jaws 2456a, 2456b clamp onto the collar 2447, clamping portions 2476a, 2476b of the collar 2447 can clamp onto a cannula positioned in an opening 2481. The clamp assembly 2454 can include, without limitation, linkage assemblies, locking mechanisms, joints, hinges, combinations thereof, or the like. The configuration and components of the clamp assembly 2454 can be selected based on the procedure to be performed.

The latch mechanism 2460 includes a lever 2480 and a link 2482. When the lever 2480 is in a closed position (FIG. 44), the clamp assembly 2454 holds the collar 2447. As the lever 2480 is moved towards the open position (FIGS. 45 and 46), the lever 2480 causes rotation of the jaw 2456a about an axis of rotation 2488 defined by a pin 2490. The link 2482 is coupled to the lever 2480 by a pin 2494. The link 2482 is coupled to the jaw 2456a by a pin 2495. The pins 2495, 2494 define axes of rotation 2502, 2504, respectively. A pin 2510 couples the lever 2480 to the jaw 2456b and defines an axis of rotation 2520. Other types of latch mechanisms can have different configurations and components (e.g., pins, levers, handles, biasing devices, etc.).

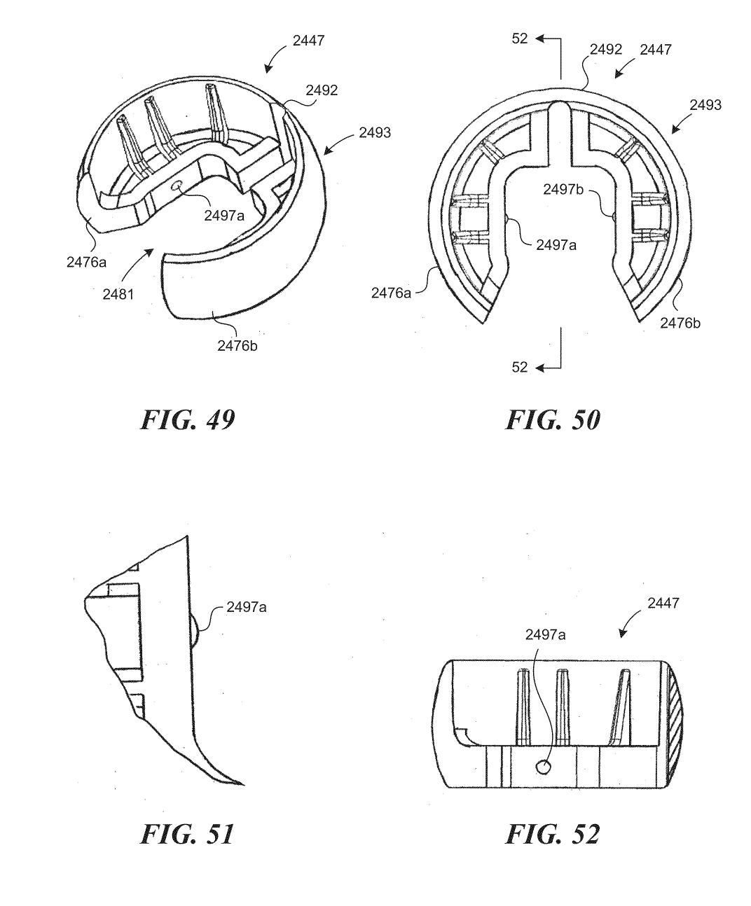

FIG. 49 is an isometric view of the collar 2447. FIG. 50 is a top view of the collar 2447. FIG. 51 is a detailed view of a portion of the collar 2447. FIG. 52 is a cross-sectional view of the collar 2447 taken along line 52-52 of FIG. 50. Referring to FIGS. 49 and 50 together, the collar 2447 includes a flexure portion 2492 and a holder portion 2493. The flexure portion 2492 allows the holder portion 2493 to expand when a cannula is moved into the opening 2481. When the cannula is positioned in the opening 2481, the flexure portion 2492 can bias the expanded holder portion 2493 towards an unexpanded configuration, thereby clamping onto the cannula.

The holder portion 2493 can include clamping portions 2476a, 2476b. The clamping portions 2476a, 2476 include keying features 2497a, 2497b. The keying features 2497a, 2497b can be similar or identical to one another and, thus, the description of one keying feature applies equally to the other, unless indicated otherwise. Referring to FIGS. 51 and 52, the keying feature 2497a is configured to engage the cannula to minimize, limit, or substantially prevent movement (e.g., axial movement) of the cannula. In some embodiments, the keying feature 2497a is a protrusion that can be received by a complementary shaped keying feature (e.g., a recess 2441 of FIGS. 40 and 41) of the cannula. FIG. 51 shows the keying feature 2497a in the form of a partially spherical bump. In other embodiments, the keying feature 2497a can be a protrusion, a recess, a hole, or the like. The number, configurations, and locations of the keying feature(s) can be selected based on the configurations and features of the cannula.

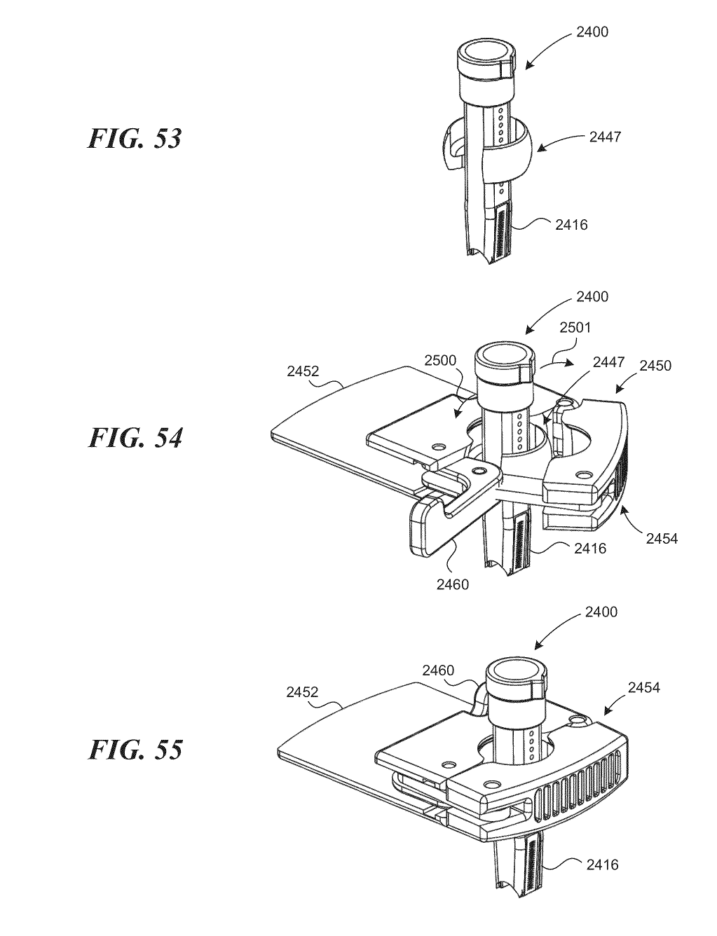

FIGS. 53-55 illustrate a method of assembling an instrument positioner assembly in accordance with an embodiment of the disclosure. Generally, the cannula 2400 can be installed in the collar 2447. The collar 2447 can be positioned in the open clamp assembly 2454. The cannula 2400 can be rotated relative to the clamp assembly 2454. After the cannula 2400 is at the desired orientation, the clamp assembly 2454 can be closed to securely hold the collar 2447. Instruments can be delivered through the cannula 2400 to access the treatment site. The clamp assembly 2454 can be opened to reorient the cannula 2400. Non-limiting exemplary methods of using the cannula 2400 and instrument positioner assembly 2450 are discussed below.

A patient can be placed on a radiolucent table in the prone and flexed position. A treatment level and accurate midline position can be determined using, for example, a needle (e.g., a spinal needle), dilator, surgical instrument (e.g., scalpel), and/or imaging. After identifying the target surgical level, an incision (e.g., a 12 mm-15 mm midline incision) can be made at the treatment level using a surgical instrument. Tissue can be separated along the midline of the supraspinous ligament. A longitudinal stab incision can be formed generally along the midline of the supraspinous ligament to preserve the supraspinous ligament. One or more dilation instruments can be used to dilate the interspinous space. Visualization (e.g., lateral fluoroscopy) can be used to ensure that the dilation instruments do not damage or traumatize non-targeted tissue.

A user can select a desired axial position of the twelve axial positions along the cannula 2400 for the collar 2447 based on, for example, the distance from the patient's skin to the treatment site. The collar 2447 can be snapped onto the cannula 2400 such that the keying feature 2497a of the collar 2447 is received by the keying feature 2441 of the cannula 2400. FIG. 53 shows the collar 2447 coupled to the cannula 2400 and ready for installation in the clamp assembly 2454. The cannula 2400 can be inserted into the patient before or after installing the collar 2447.

The instrument positioner apparatus 2450 can be placed over the collar 2447. The base 2452 can rest against the patient's skin and can extend in the superior direction. The cannula 2400 can be rotated in the lateral direction (indicated by arrows 2500, 2501) or other desired direction. The base 2452 can inhibit or limit rocking movement of the instrument positioner apparatus 2450 in the superior direction, thereby stabilizing the cannula 2400.

The lever mechanism 2460 can be used to close the clamp assembly 2454. FIG. 55 shows the closed clamp assembly 2454 holding collar 2447. The cannula 2400 is keyed to the collar 2447 to prevent axial movement of the cannula 2400. The clamp assembly 2454 can be opened to adjust the orientation of the cannula 2400. When an instrument is positioned in the cannula 2400, a depth stop mechanism of the instrument can contact the cannula 2600 to limit movement of the instrument in the distal direction.

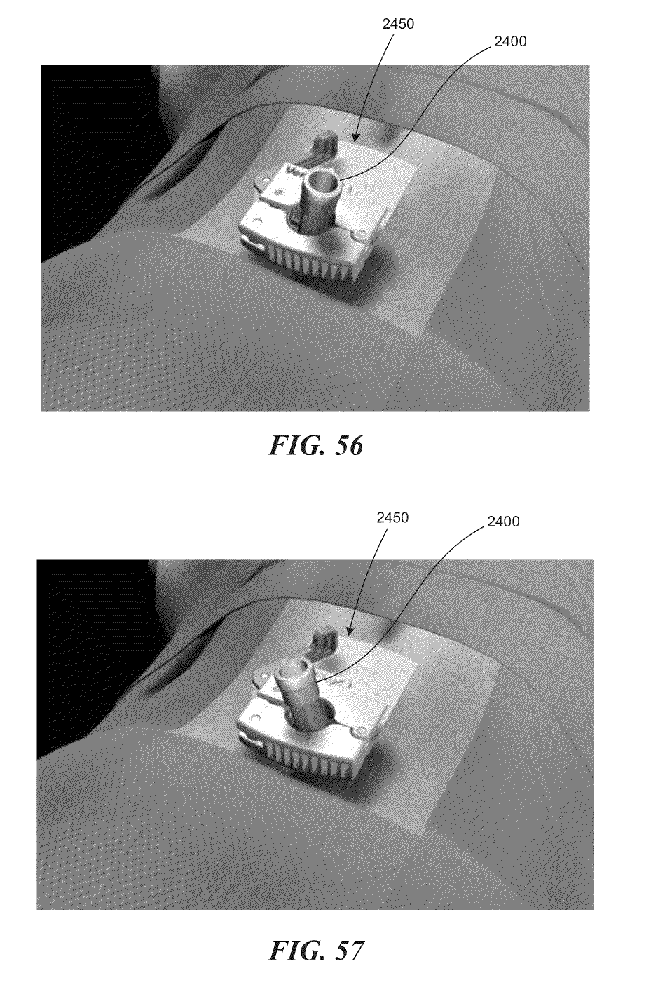

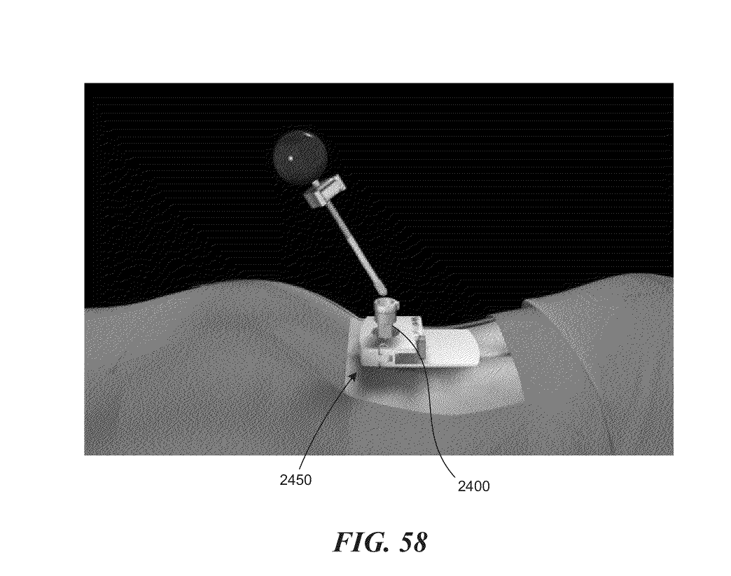

FIGS. 56-58 illustrate the instrument positioner assembly 2450 positioned on a patient. The cannula 2400 of FIG. 56 is positioned to access left regions of the subject's left lateral vertebrae recess of a vertebral body. The cannula 2400 of FIG. 57 is positioned to access the right regions of the subject's right lateral recess of the vertebral body. FIG. 58 shows a reamer instrument ready to be delivered through the cannula 2400. Visualization techniques can be used to confirm the position, trajectory, and depth of the end of the reamer instrument. In some procedures, the reamer instrument can extend 15 mm past the distal end of the cannula 2400 when a stop member contacts the cannula 2400. The reamer instrument can be rotated to abrade, loosen, tear, or otherwise alter tissue. The reamer instrument can be removed any number of times to remove residual tissue (e.g., ligament tissue, bone tissue, etc.) attached to the reamer instrument. Reamer instruments can be used to cut bone, create one or more defects (e.g., a generally hemispherical defect) in the inferior medial aspect of the superior lamina, or otherwise prepare the treatment site.

To remove midline tissue, the cannula 2400 can be oriented towards the midline interlaminar region. A reamer instrument can be inserted through the cannula 2400 and positioned towards the midline position of the superior lamina. The depth stop mechanisms can be used to, for example, prevent injury to the dural or other non-targeted tissue. Visualization techniques can be used to monitor the position on the reamer head. In some procedures, the reamer head can be moved from midline to left lateral or the right lateral. Any number of reamer instruments can be used to remove the desired amount of midline lamina bone. The depth stop mechanism can be used to allow access to the targeted region while maintaining a desired distance from the epidural space and other vital structures. After performing the reaming procedure, the reamer can be removed from the patient and a preparation procedure can be performed. The preparation procedure can include, without limitation, irrigating the treatment site, removing residual tissue (e.g., via suction), applying one or more agents (e.g., hemostatic agents), or other procedures.

A debulker instrument can be used to provide a complete blunt dissection of the ligamentum flavum from the lamina and disrupt ligamentous tissue. For example, the debulking tip of FIG. 33A has cutting edges 2121a, 2121b to cut tissue when the debulking head 2119 is moved in the lateral direction. The engagement regions 2123, 2125 can be pressed against the tissue while the debulking head 2119 is moved to cut, roughen, dislodge, or otherwise alter tissue at the treatment site.

In some procedures, the debulker instrument is inserted through the cannula 2400 and positioned at a midline position of the superior lamina. The depth stop mechanisms can facilitate positioning of the distal tip (e.g., debulking head) at the most dorsal margin of the superior lamina. Intraoperative fluoroscopy and/or tactile feedback can be used to confirm positioning. While maintaining a midline trajectory, the distal tip of the debulker instrument can be gently moved around the inferior lamina lip and repositioned against the bony underside. The adjustable depth stop can be reset, if desired, to allow access to the targeted region while maintaining a desired distance from the epidural space and other vital structures. The properly positioned distal tip can engage the underside of the lamina and resist attempts to gently withdraw the instrument.

The debulking tip can dissect and separate the ligamentum flavum from the lamina when it is move from midline toward the lateral recess. A subtle left-right sweeping motion can be used disrupt ligamentous tissue and help extend the desired tissue plane. The distal tip can be moved until it reaches the most lateral desired position. The depth stop mechanism can be adjusted to allow access to the lateral recesses. The debulker tip can be moved slightly inferior and out from the lamina underside. The debulker tip can be used to continually debulk the ligamentum flavum. The depth stop mechanism can be adjusted to allow access to the targeted region, while intraoperative fluoroscopy is used to verify the distal tip position and maintain a safe working distance from the epidural space and/or other vital structures. After performing the debulking procedure, the debulker instrument can be removed from the patient and a preparation procedure can be performed.

The lamina can be removed using a tissue removal instrument. The cannula 2400 can be oriented towards the desired interlaminar region (e.g., left or right interlaminar region). A closed jaw assembly of a tissue removal instrument can be moved through the cannula 2400 towards a generally midline position. The depth stop mechanism can be used to adjust the depth of penetration until the jaw assembly is positioned proximate the most dorsal margin of the superior lamina. The jaw assembly can be closed to remove tissue. While maintaining midline trajectory, the jaw assembly can be moved around the inferior lamina lip and positioned against the bony underside. The depth stop can be adjusted to allow access to the targeted region while maintaining a desired distance from the epidural space and other vital structures. The distal or lower jaw of the jaw assembly can engage the underside of the lamina and the proximal or upper jaw can be positioned just dorsal to the lamina. The jaw assembly can be held against the targeted lamina bone while the jaw assembly is closed. The tissue removal instrument can be withdrawn from the patient. The jaw assembly can be opened to release the captured material. This process can be repeated to remove bone and other tissue in the lateral direction until the desired decompression is achieved.

Systems, components, and instruments disclosed herein can be disposable or reusable. For example, the tool 2110 of FIGS. 31-33 can be disposable to prevent cross-contamination. As used herein, the term “disposable” when applied to a system or component (or combination of components), such as an instrument, a tool, or a distal tip or a head (e.g., a reamer head, a jaw assembly, etc.), is a broad term and generally means, without limitation, that the system or component in question is used a finite number of times and is then discarded. Some disposable components are used only once and are then discarded. In other embodiments, the components and instruments are non-disposable and can be used any number of times. For example, the actuator mechanism 1010 (FIGS. 26-28) may be non-disposable and subjected to different types of cleaning and/or sterilization processes and the tissue removal device 1012 (FIGS. 26-29) can be disposable.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. For example, visualization media can be delivered before, during, or after positioning a cannula (e.g., cannula 130 of FIG. 14). Thus, the act 424 of FIG. 11 can be performed before the act 420 of FIG. 11. Additionally, the instruments (e.g., tissue removal instrument, reamer instrument, debulker instrument, dilator, syringe, etc.) can have one or more stops (e.g., depth stops) to inhibit or prevent injury or damage to tissue. Additionally or alternatively, the stops can be incorporated into the cannulas (e.g., cannula 130). The various embodiments described herein may also be combined to provide further embodiments. For example, features from various instruments can be combined with features disclosed in U.S. Pat. No. 8,012,207; U.S. Pat. No. 8,123,807; U.S. Pat. No. 8,152,837, and U.S. application Ser. No. 12/217,662 (U.S. Publication No. 2008/0287997), which are hereby incorporated by reference herein and made a part of this application.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method for treating spinal nerve compression of a human subject, comprising:
positioning a media delivery device between a first vertebra and a second vertebra;
delivering a visualization media from an outlet of the media delivery device while the outlet of the media delivery device is positioned outside of an epidural space between a spinal cord and a ligamentum flavum of the human subject; and
removing tissue at a treatment site using an instrument while viewing the instrument and the delivered visualization media.

2. The method of example 1 wherein delivering the visualization media includes delivering the visualization media from the outlet which is positioned at a delivery site, the delivery site is between a dural sac and nerve tissue of the spinal cord or within the ligamentum flavum.

3. The method of example 1 wherein delivering the visualization media from the outlet includes delivering at least most of the visualization media from the outlet while the outlet is positioned outside of the epidural space.

4. The method of example 1 wherein delivering the visualization media from the outlet of the delivery device includes delivering the visualization media through the outlet while a portion of the delivery device is positioned between the first vertebra and the second vertebra.

5. The method of example 1 wherein removing tissue at the treatment site includes removing bone from the spine of the human subject, separating a portion of the ligamentum flavum from the vertebra, and/or removing a portion of the ligamentum flavum.

6. The method of example 1, further comprising viewing the instrument and the visualization media via fluoroscopy.

7. A method for treating spinal cord compression, comprising:
injecting a visualization media into tissue outside a region of an epidural space located between a spinal cord and a ligamentum flavum of the subject; and
removing tissue using a tissue removal instrument while viewing the tissue removal instrument and the delivered visualization media.

8. The method of example 7, further comprising viewing at least one non-targeted anatomical structure using the delivered visualization media while removing the tissue.

9. The method of example 8 wherein the non-targeted anatomical structure is the spinal cord.

10. The method of example 7 wherein removing the tissue using the tissue removal instrument includes loosening tissue and removing the loosened tissue from the subject to reduce spinal cord compression.

11. A system, comprising:
an instrument positioner assembly including:
a base configured to be positioned on a human subject;
a cannula having a first end, a second end, and an instrument delivery passageway extending between the first end and the second end; and
a joint device configured to rotatably couple the cannula to the base such that the first end of the cannula is positionable between a first spinous process of a first vertebra and a second spinous process of a second vertebra.

12. The system of example 11 wherein the joint device includes a collar, and the base includes a socket configured to rotatably hold the collar.

13. The system of example 12 wherein the collar includes a partially spherical surface configured to slidably engage a partially spherical surface of the socket.

14. The system of example 12 wherein the cannula includes a plurality of spaced apart keying features, and the collar includes at least one keying feature configured to engage one of the keying features of the cannula.

15. The system of example 12 wherein the collar has a flexure portion and a holder portion, wherein the holder portion defines a receiving opening, and the flexure portion allows the holder portion to expand when the cannula is moved into the receiving opening.

16. The system of example 15 wherein the flexure portion biases the holder portion in the expanded configuration towards an unexpanded configuration to hold the cannula.

17. The system of example 11 wherein the base includes a clamp assembly movable between a closed configuration for fixedly holding the cannula and an open configuration for allowing rotation of the cannula.

18. The system of example 11 wherein the joint device includes a linkage assembly coupled to the base.

19. The system of example 11 wherein the cannula defines a plurality of discrete axial mounting positions for the collar.

20. The system of example 11, further comprising:
a tissue removal instrument configured to be delivered along the instrument delivery passageway while the joint device holds the cannula.

21. The system of example 11, further comprising:
a debulker instrument configured to be delivered along the instrument delivery passageway.

22. The system of example 11, further comprising:
a media delivery device holding a visualization media, wherein the media delivery device includes a needle configured to pass between the first vertebra and the second vertebra and to deliver the visualization media within the vertebral foremen.

23. The system of example 11, further comprising:
a media delivery device including a needle configured to pass between the first vertebra and the second vertebra and to deliver the visualization media at a location between a first vertebral foramen of the first vertebra and a second vertebral foramen of the second vertebra.

24. The system of example 11, further comprising:
a syringe holding the visualization media and including a 26 gauge needle.

25. A method of treating spinal nerve compression at a target site of a spine of a patient, comprising:
delivering a visualization media from an outlet of a media delivery device positioned within the dural sac such that the visualization media is retained within the dural sac and contacts the spinal cord; and
removing tissue at the target site using a tissue removal instrument while viewing the tissue removal instrument and the visualization media within the dural sac.

26. The method of claim 25 wherein removing the tissue includes removing tissue from a first lateral recesses of a vertebra and removing tissue from a second lateral recess of the vertebra using the instrument.

27. The method of claim 26, further comprising repositioning a cannula in the patient to provide access to the first and second lateral recesses.

28. A method of treating spinal nerve compression at a target site of a spine of a patient, comprising:
delivering a visualization media to the patient;
removing tissue at the target site using a tissue removal instrument while viewing the tissue removal instrument and the visualization media; and
delivering a spinal implant into the patient.

29. The method of claim 28, further comprising viewing the spinal implant and the delivered visualization media after removing the tissue.

30. A surgical instrument, comprising:
a tool having a main body and a distal portion; and
a handle assembly including a handle and a depth stop mechanism, wherein the handle assembly is configured to be manually gripped by a user, and wherein the depth stop mechanism is movable to adjust a depth of penetration of the distal portion of the tool.

31. The surgical instrument of claim 30 wherein the depth stop mechanism includes a locking assembly and a stop member, wherein the locking assembly has a locked configuration for holding the stop member relative to the main body and an unlocked configuration for moving the stop member along the main body.

32. The surgical instrument of claim 31 wherein the stop member includes a head that surrounds the main body, the head is movable axially along a longitudinal axis of the main body.

33. The surgical instrument of claim 31 wherein the locking assembly has a control element movable from an undepressed position to a depressed position to move the depth stop mechanism from the locked configuration to the unlocked configuration.

34. The surgical instrument of claim 30 wherein the depth stop mechanism includes a stop member and a locking assembly, wherein the stop member includes a biasing element that urges the locking assembly towards a locked configuration, wherein a user can overcome a biasing force provided by the biasing element to move the depth stop mechanism from the locked configuration to the unlocked configuration.

35. The surgical instrument of claim 30 wherein the depth stop mechanism includes a stop member and a locking assembly, wherein the stop member includes a biasing element that urges the locking assembly towards a locked configuration, wherein a user can overcome a biasing force provided by the biasing element to move the depth stop mechanism from the locked configuration to the unlocked configuration.

36. The surgical instrument of claim 30 wherein the depth stop mechanism includes a stop member slidably positionable along the main body, wherein the depth stop mechanism is movable between a locked configuration for holding the stop member stationary relative to the main body and an unlocked configuration for allowing the stop member to slide along the main body.

37. The surgical instrument of claim 30 wherein the distal portion includes cutting edges configured to cut tissue.

38. The surgical instrument of claim 30 wherein the distal portion includes a jaw assembly movable from an open configuration to a closed configuration to capture tissue.

39. The surgical instrument of claim 38 wherein the handle assembly includes a lever operable to move the jaw assembly from the open configuration to the closed configuration.

40. The surgical instrument of claim 30 wherein the distal portion is a reamer head.

41. The surgical instrument of claim 30 wherein the distal portion includes a tissue debulker.

42. A surgical instrument for a spinal decompression procedure, comprising:
a tool having a main body and a distal portion; and
a handle assembly including a handle and a depth stop mechanism movable along the tool to adjust a depth of penetration of the distal portion of the tool when altering tissue along a subject's spine using the tool.

43. The surgical instrument of claim 42 wherein the depth stop mechanism includes a locking assembly and a stop member, wherein the locking assembly has a locked configuration for holding the stop member relative to the main body and an unlocked configuration for moving the stop member along the main body.

44. The surgical instrument of claim 42 wherein the distal portion includes a jaw assembly movable from an open configuration for receiving tissue to a closed configuration for holding tissue.

45. The surgical instrument of claim 44 wherein the handle assembly includes a lever operable to move the jaw assembly from the open configuration to the closed configuration.

46. The surgical instrument of claim 42 wherein the tool is a reamer.

47. The surgical instrument of claim 42 wherein the distal portion includes a tissue debulker.
